SEQUENCE LISTING (6 sequences)

SEQ ID NO: 1 - protein, 195 aa, synthetic construct
  DISULFID 157: note = Interchain disulfide bond with the cysteine at position 427 of SEQ ID NO: 2
AQQGEEDPQALSIQEGENATMNCSYKTSINNLQWYRQNSGRGLVHLILIRSNEREKHSGRLRVTLDTSKKSSSLLITASRAADTASYFCATDGSTPMQFGKGTRLSVIANIQKPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 2 - protein, 500 aa, synthetic construct
  DISULFID 427: note = Interchain disulfide bond with the cysteine at position 157 of SEQ ID NO: 1
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSWAQGDFQKGDIAEGYSVSREKKESFPLTVTSAQKNPTAFYLCASSWGAPYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 3 - protein, 9 aa, Homo sapiens
YLEPGPVTA

SEQ ID NO: 4 - protein, 201 aa, synthetic construct
  DISULFID 163: note = Interchain disulfide bond with the cysteine at position 429 of SEQ ID NO: 5
GDAKTTQPNSMESNEEEPVHLPCNHSTISGTDYIHWYRQLPSQGPEYVIHGLTSNVNNRMASLAIAEDRKSSTLILHRATLRDAAVYYCILILGHSRLGNYIATFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 5 - protein, 502 aa, synthetic construct
  DISULFID 429: note = Interchain disulfide bond with the cysteine at position 163 of SEQ ID NO: 4
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSDGGITQSPKYLFRKEGQNVTLSCEQNLNHDAMYWYRQDPGQGLRLIYYSQIMGDEQKGDIAEGYSVSREKKESFPLTVTSAQKNPTAFYLCASSWWTGGASPIRFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 6 - protein, 9 aa, Homo sapiens
SLLQHLIGL

(19) United States
(12) Patent Application Publication
(10) Pub. No.: US 2026/0357348 A1
(43) Pub. Date: Nov. 12, 2026

(54) COMPOSITIONS AND METHODS FOR TREATING CANCER THAT DEMONSTRATES DECREASING LEVELS OF CTDNA IN A SUBJECT

(71) Applicants: IMMUNCORE LIMITED, Abingdon, Oxfordshire (GB); IMMUNOCORE LLC, Rockville, MD (US)

(72) Inventors: Koustubh RANADE, Gaithersburg, MD (US); Laura COLLINS, Abingdon, Oxfordshire (GB)

(21) Appl. No.: 19/108,120

(22) PCT Filed: Aug. 31, 2023
(86) PCT No.: PCT/US2023/031724

§ 371 Date: Feb. 28, 2025

(30) Foreign Application Priority Data
Aug. 31, 2022 (WO) PCT/US2022/075791

(51) Int. Cl. C12Q 1/6886; A61K 35/17; A61K 40/11; A61K 40/32; A61K 40/42; A61P 35/00; C07K 14/705; C12N 5/0783

(52) U.S. Cl. CPC C12Q 1/6886; A61K 35/17; A61K 40/11; A61K 40/32; A61K 40/4224; A61P 35/00; C07K 14/7051; C12N 5/0636; A61K 2239/15; A61K 2239/49; A61K 2239/57; C12N 2510/00; C12Q 2600/106; C12Q 2600/156; C12Q 2600/16

(57) ABSTRACT

The present disclosure relates to the treatment of a cancer in a human subject where the treatment includes continuing administration of a T cell receptor (TCR) therapy in subjects whose ctDNA levels do not exceed baseline following administration of a first plurality of doses of TCR therapy. In particular, the present disclosure relates to treatment of a gp100 positive cancer in a human subject where the treatment includes administering tebentafusp to a subject who has or is suspected of having a gp100 positive cancer or treatment of a FRAME positive cancer in a human subject where the treatment includes administering a PRAME-targeting soluble bispecific TCR therapeutic (IMC-F106C) to a subject who has or suspected of having a FRAME positive cancer. The treatment includes continuing administration of tebentafusp or IMC-F106C in subjects whose ctDNA levels do not exceed baseline following administration of a first plurality of doses of tebentafusp or IMC-F106C.

### 1. CROSS-REFERENCE TO RELATED APPLICATIONS

This application claims the benefit of and priority to International Application No. PCT/US2022/075791, filed Aug. 31, 2022, which is hereby incorporated by reference in its entirety.

### 2. SEQUENCE LISTING

The instant application contains a Sequence Listing, which has been submitted as an XML file on triplicate CD-Rs (labeled as CRF, Copy 1, and Copy 2) and mailed on Aug. 31, 2023 via USPS Priority Mail Express; and is hereby incorporated by reference in its entirety. Said XML copy, created on Oct. 21, 2022, is named 56638WO_CRF_sequencelisting.xml, and is 9,158 bytes in size.

### 3. BACKGROUND

The human glycoprotein 100 (gp100) is one of a panel of melanoma-associated antigens to which the body can mount a natural immune response. The protein is a 661 amino acid melanosomal membrane-associated glycoprotein which is expressed in normal melanocytes and widely overexpressed on the majority of melanoma cancer cells. For example, one study found that 82% of 192 melanoma metastases from 28 melanoma patients expressed gp100 (Trefzer et al., Melanoma Res. 16, 137-45 (2006)). Several studies have reported higher expression levels of gp100 in melanoma tissues (Hofbauer et al., J Immunother. 27, 73-78 (2004); Barrow et al., Clin Cancer Res. 12:764-71 (2006)). The exact function of the protein is unknown but it appears to be involved in melanosome maturation (Hoashi T, et al., J Biol Chem 280, 14006-14016 (2005); Kawakami Y and Rosenberg S A, Int Rev Immunol 14, 173-192 (1997)).

The development of tebentafusp (i.e., approved product KIMMTRAK® (tebentafusp-tebn)), a bispecific gp100 peptide HLA-A*02:01 directed T cell receptor—CD3 T cell engager provided a new treatment option for melanoma patients (e.g., metastatic uveal melanoma). The T cell receptor (“TCR”) arm binds to a gp100 peptide presented by human leukocyte antigen-A*-2:01 (HLA A*02:01) on the cell surface of melanoma tumor cells. The mechanism of action of such a therapeutic is significantly different to other immunotherapy predecessors and results in the rapid and potent redirection of non-gp100 specific T cells to kill gp100 positive cells in vitro.

PRAME, or Preferentially Expressed Antigen In Melanoma, was first identified as an antigen that is over expressed in melanoma (Ikeda et al, Immunity 6, 199-208 (1997); it is also known as CT130, MAPE, OIP-4 and has Uniprot accession number P78395. PRAME belongs to the family of germline-encoded antigens known as cancer testis antigens. Cancer testis antigens are attractive targets for immunotherapeutic intervention since they typically have limited or no expression in normal adult tissues. PRAME is expressed in a number of solid tumors as well as in leukemias and lymphomas (Doolan et al, Breast Cancer Res Treat. 109, 359-65 (2008) Epping et al, Cancer Res. 66, 10639-42 (2006); Ercolak et al, Breast Cancer Res Treat. 109, 359-365 (2008); Matsushita et al, Leuk Lymphoma. 44, 439-44 (2003); Mitsuhashi et al, Int. J Hematol. 100, 88-95 (2014); Proto-Sequeire et al, Leuk Res. 30, 1333-9 (2006); Szczepanski et al, Oral Oncol. 49, 144-51 (2013); Van Baren et al, Br J Haematol. 102, 1376-9 (1998).

The peptide SLLQHLIGL (SEQ ID NO: 6) corresponds to amino acids 425-433 of the full length PRAME protein and is presented on the cell surface in complex with HLA-A*02 (Kessler et al., J Exp Med. 2001 Jan. 1; 193 (1): 73-88). This peptide-HLA complex provides a useful target for TCR-based immunotherapeutic intervention.

IMC-F106C is a T cell redirecting bispecific therapeutic agent comprising a soluble affinity enhanced TCR that binds to the SLLQHLIGL (SEQ ID NO: 6) peptide-HLA-A*02 complex, fused to an anti-CD3 scFv. The targeting end of IMC-F106C (the soluble TCR) binds to a peptide fragment of the PRAME antigen presented by HLA-A*02 on the surface of cancer cells. The effector end of IMC-F106C (anti-CD3 scFv) can bind to CD3 on any T cell, redirecting the T cell to produce effector cytokines and/or kill the cell presenting the target. Even with the development of tebentafusp and IMC-F106C, there remains a need to improve overall survival of patients with gp100 positive and PRAME positive cancers and determine patients most likely to benefit from continued treatment with tebentafusp and/or IMC-F106C.

### 4. SUMMARY

Provided herein are methods of treating cancer in a subject, comprising (a) measuring the circulating tumor DNA (ctDNA) level in the subject prior to administering a T cell therapy to obtain a baseline ctDNA level; (b) administering to the subject a first plurality of doses of the T cell therapy; (c) measuring the ctDNA level in the subject following administration of the first plurality of doses of the T cell therapy to obtain a post-treatment ctDNA level; and (d) administering at least a second plurality of doses of a T cell therapy when the post-treatment ctDNA level is less than or equal to the baseline ctDNA level.

In some embodiments, the subject has a tumor that is larger in size following administration of the first plurality of doses of the T cell therapy compared to the size of the tumor prior to administration of the T cell therapy, as measured using radiographic analysis.

In some embodiments, the cancer is positive for gp100 and/or PRAME. In some embodiments, the cancer is selected from the group consisting of: melanoma, ovarian cancer, non-small cell lung adenocarcinoma, non-small cell lung squamous cell carcinoma, and breast cancer. In some embodiments, the cancer is uveal melanoma or cutaneous melanoma. In some embodiments, the cancer is triple-negative breast cancer. In some embodiments, the cancer is metastatic.

In some embodiments, the subject is determined to have progressive disease (PD) according to RECIST criteria following administration of at least the first plurality of doses of the T cell therapy.

In some embodiments, the subject is determined to have stable disease (SD) according to RECIST criteria following administration of at least the first plurality of doses of the T cell therapy.

In some embodiments, the T cell therapy is tebentafusp. In some embodiments, the first plurality of doses comprises at least one starting dose of tebentafusp in the range of 10 to 30 micrograms. In some embodiments, the first plurality of doses further comprises at least one escalated dose of tebentafusp in the range of 20 to 40 micrograms, wherein the escalated dose is higher than the starting dose and is administered after the at least one starting dose. In some embodiments, the first plurality of doses further comprises at least one continuing dose of tebentafusp of at least 50 micrograms administered after the at least one escalated dose. In some embodiments, the continuing dose is 68 micrograms.

In some embodiments, the T cell therapy is IMC-F106C. In some embodiments, the first plurality of doses comprises at least one starting dose of IMC-F106C in the range of 5 to 40 micrograms. In some embodiments, the first plurality of doses further comprises at least one escalated dose of IMC-F106C in the range of 15 to 80 micrograms, wherein the escalated dose is higher than the starting dose and is administered after the at least one starting dose. In some embodiments, the first plurality of doses further comprises at least one continuing dose of IMC-F106C in the range of 60 to 400 micrograms, wherein the continuing dose is higher than the escalated dose and is administered after the at least one escalated dose.

In some embodiments, the first plurality of doses comprises at least one treatment cycle, wherein a treatment cycle consists of once-weekly doses of the T cell therapy for a period of three weeks (21 days) or four weeks (28 days).

In some embodiments, the first plurality of doses consists of four doses administered once weekly and wherein the post-treatment ctDNA level is measured at week 5 following administration of the first dose of T cell therapy. In some embodiments, ctDNA is measured by amplifying a panel of genes and then sequencing the amplicons to identify mutations. In some embodiments, ctDNA is measured by amplification of a panel of tumor-specific gene mutations from samples of serum from the subject. In some embodiments, the amplified ctDNA is assessed to confirm the presence of mutations in a specific group of genes. In some embodiments, the amplified ctDNA is assessed for mutations in a group of genes consisting of GNAQ, GNA11, SF3B1, PLCB4, CYSLTR2 and EIF1AX.

In some embodiments, the post-treatment ctDNA level is reduced by at least 50%, at least 60%, at least 65%, or at least 68% from baseline.

In another aspect, disclosed herein are methods of treating melanoma in a subject comprising administering to the subject a first dose of 20 micrograms tebentafusp, a second dose of 30 micrograms tebentafusp, a third dose of at least 50 micrograms of tebentafusp, and a fourth dose of at least 50 micrograms of tebentafusp, and as long as ctDNA levels remain stable or are reduced as compared to a baseline ctDNA level, subsequent weekly doses of at least 50 micrograms tebentafusp, wherein doses are administered every 6 to 8 days. In some embodiments, the melanoma is cutaneous melanoma or uveal melanoma.

In another aspect, disclosed herein are methods of treating a PRAME positive cancer in a subject comprising administering to the subject a first dose of IMC-F106C in the range of 5 to 40 micrograms, a second dose of IMC-F106C in the range of 15 to 80 micrograms, and a third dose of IMC-F106C in the range of 60 to 400 micrograms, and as long as ctDNA levels remain stable or are reduced as compared to a baseline ctDNA level, subsequent weekly doses of IMC-F106C in the range of 60 to 400 micrograms, wherein the second dose is higher than the first dose and the third dose is higher than the second dose and wherein the doses are administered every 6 to 8 days. In some embodiments, the PRAME positive cancer is selected from the group consisting of melanoma, ovarian cancer, non-small cell lung adenocarcinoma, non-small cell lung squamous cell carcinoma, and breast cancer. In some embodiments, the PRAME positive cancer is uveal melanoma or cutaneous melanoma. In some embodiments, the PRAME positive cancer is triple-negative breast cancer.

In another aspect, disclosed herein are methods of treating a subject who has or is suspected of having uveal melanoma, comprising (a) administering to the subject a first plurality of doses of tebentafusp or IMC-F106C; (b) determining that the subject has a reduction in ctDNA from baseline following administration of the first plurality of doses of tebentafusp or IMC-F106C, wherein the reduction in ctDNA is determined by evaluating variant allele frequency in a panel of mutations; and (c) administering to the subject at least a second plurality of doses of tebentafusp or IMC-F106C. In some embodiments, the panel of mutations comprises GNAQ (Q209L/P); GNA11 (Q209L); SF3B1 (K700E, R625L/H/C); PLCB4 (D630N/Y/V); CYSLTR2 (L129Q); and EIF1AX (G15D).

In another aspect, disclosed herein are kits comprising (a) at least one solution for use in processing a sample obtained from a human subject, wherein the sample comprises cell-free DNA; and (b) nucleotide primers for evaluating variant allele frequency in a panel of mutations comprising GNAQ (Q209L/P); GNA11 (Q209L); SF3B1 (K700E, R625L/H/C); PLCB4 (D630N/Y/V); CYSLTR2 (L129Q); and EIF1AX (G15D).

### 5. BRIEF DESCRIPTION OF THE DRAWINGS

These and other features, aspects, and advantages of the present invention will become better understood with regard to the following description, and accompanying drawings, where:

FIG. 1 shows the percentage of treated patients experiencing grade 1-2 or grade 3-4 treatment-related adverse events after each dose of tebentafusp. Number of patients receiving dose 1=127, dose 2=122, dose 3=122, dose 4=119, and dose 8=113.

FIG. 2 is a Kaplan-Meier plot of overall survival (n=127). Events are deaths due to any cause. Patients not known to have died at the time of analysis were censored. Median overall survival was 16.8 (95% CI: 12.9-21.3) months with a 1-year overall survival rate of 62% (95% CI: 53-70%)

FIG. 3A is a waterfall plot showing log 10 change in circulating tumor DNA (ctDNA) level by week 9 in all evaluable patients (n=94).

FIG. 3B shows correlation between circulating tumor DNA (ctDNA) reduction in metastatic uveal melanoma (mUM) patients by week 9 with tebentafusp and hazard ratio for death (HR). Hazard ratios were derived by comparing subsets of patients with ctDNA above or below the thresholds indicated on the x-axis.

FIG. 3C is a Kaplan-Meier comparison of overall survival of patients with ctDNA clearance (n=12) versus patients without clearance (n=82) by 9 weeks (HR 0.08; 95% CI 0.01-0.54, p=0).

FIG. 3D is a Kaplan-Meier comparison of overall survival in patients with best overall response of progressive disease with a reduction in ctDNA by ≥0.5 log fold change (n=16) versus <0.5 log fold change (n=31) by week 9. Hazard ratio for death 0.47 (95% CI 0.22-1.01), p=0.042.

FIG. 3E shows correlation between ctDNA reduction and overall survival in patients with metastatic uveal melanoma treated with tebentafusp.

FIG. 4 is a Kaplan-Meier plot of progression-free survival for patients treated with tebentafusp. Tick marks represent patients who were known to be alive and without disease progression as assessed by Response Evaluation Criteria in Solid Tumors (RECIST), version 1.1. Median progression-free survival was 2.76 months (95% CI 2-3.7).

FIG. 5 shows level of circulating tumor DNA (ctDNA) at baseline plotted against tumor size at baseline (n=94). Tumor size was measured as the sum of the longest diameters of the target lesions at first scan according to RECIST v1.1.

FIG. 6 shows data indicating the presence of specific mutations in GNAQ, GNA11, PLCB4, and SF3B1 in baseline ctDNA and tumor biopsies from patients in which both sets of data were available (n=45).

FIG. 7 shows data demonstrating that a reduction in ctDNA was observed across tumor types in subjects treated once weekly with IMC-F106C for eight weeks. Abbreviations are as follows: B, triple-negative breast cancer; CPI, checkpoint inhibitor; C, cutaneous melanoma; ctDNA, circulating tumor DNA; E, endometrial carcinoma; LA, non-small cell lung adenocarcinoma; LS, non-small cell lung squamous cell carcinoma; O, ovarian cancer; U, uveal melanoma; PD, progressive disease; PR, partial response; SD, stable disease; NE, not evaluable; tebe, tebentafusp.

FIG. 8 is a Kaplan-Meier comparison of overall survival in patients with melanoma that had reduction in ctDNA by ≥0.5 log fold change versus <0.5 log fold change by week 9.

### 6. DETAILED DESCRIPTION

### 6.1. Definitions

Terms used in the claims and specification are defined as set forth below unless otherwise specified.

The term “ameliorating” refers to any therapeutically beneficial result in the treatment of a disease state, e.g., a melanoma, including but not limited to, increasing overall survival, reducing symptoms of disease, shrinking one or more tumors, improving response according to RECIST criteria, slowing progression, remission, or cure thereof.

The term “baseline” as used herein refers to measurements taken at the time of first administration of tebentafusp or measurements taken just prior to administration of tebentafusp.

The term “censored” as used herein refers to statistical analysis with respect to overall survival. For example, any patient not known to have died at the time of analysis is censored based on the last recorded date on which the patient was known to be alive.

The term “ctDNA clearance” as used herein refers to a reduction of ctDNA ≥99.9% compared to baseline.

The term “plurality” as used herein refers to two or more (e.g., in some embodiments, a plurality of doses is two doses; in some embodiments, a plurality of doses is three or four doses; in some embodiments, a plurality of doses is more than four doses).

The term “post-treatment” as used herein refers to a time following administration of a therapeutic. The term “post-treatment ctDNA level” as used herein refers to the ctDNA level measured in a subject following administration of a plurality of doses of T cell therapy (e.g., tebentafusup, IMC-F106C).

The term “PRAME positive cancer” as used herein refers to a cancer in which at least some of the cancer cells express PRAME.

The term “gp100 positive cancer” as used herein refers to a cancer in which at least some of the cancer cells express gp100.

The term “mammal” as used herein includes both humans and non-humans and includes but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

As used herein, the term “subject” broadly refers to a mammalian subject (e.g., a human patient).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

The term “therapeutically effective amount” is an amount that is effective to ameliorate a symptom of a disease (e.g., uveal melanoma).

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

Abbreviations used in this application include the following: best overall response (BOR); disease control rate (DCR); complete response (CR); cytokine release syndrome (CRS); objective response rate (ORR); overall survival (OS); duration of response (DOR); minor response (MR); partial response (PR); progressive disease (PD); progression-free survival (PFS); recommended phase 2 dose (RP2D); Response Evaluation Criteria in Solid Tumors (RECIST); stable disease (SD); sum of the longest diameters (SLD).

It must be noted that, as used in the specification and the appended claims, the singular forms “a,” “an” and “the” include plural referents unless the context clearly dictates otherwise.

### 6.2. T Cell Therapy

In some embodiments, T cell therapy comprises chimeric antigen receptor (CAR) T cells. In some embodiments, T cell therapy comprises soluble T cell receptors (TCRs). In some embodiments, T cell therapy comprises tumor-infiltrating lymphocytes (TILs). In particular embodiments, T cell therapy comprises a bispecific soluble T cell receptor fused to a CD3 T cell engager (e.g., tebentafusp, IMC-F106C).

### 6.2.1. Tebentafusp

In some embodiments, tebentafusp is the approved product KIMMTRAK® (tebentafusp-tebn), a bispecific gp100 peptide HLA-A*02:01 directed T cell receptor-CD3 T cell engager. The T cell receptor (“TCR”) arm binds to a gp100 peptide presented by human leukocyte antigen-A*-2:01 (HLA A*02:01) on the cell surface of uveal melanoma tumor cells. The anti-CD3 T cell engaging arm activates polyclonal T cells to release inflammatory cytokines and cytolytic proteins, which results in direct lysis of melanoma tumor cells. KIMMTRAK® is indicated for the treatment of HLA A*02:01-positive adult patients with unresectable or metastatic melanoma.

Tebentafusp is composed of an alpha chain and a beta chain. The alpha chain comprises a TCR alpha chain variable region domain and a constant region domain. The beta chain comprises a TCR beta chain variable region domain and a constant region domain where the TCR beta chain is linked to a single-chain variable fragment (“scFv”) anti-CD3 antibody at its N-terminus. The two chains are covalently bonded via a disulfide bond between the cysteine at position 157 on the alpha chain (α157) amino acid sequence of SEQ ID NO: 1 and the cysteine in position 427 on the anti-CD3 scFv beta chain (β427) of SEQ ID NO: 2 (indicated with asterisks, *, in the amino acid sequences below).

The amino acid sequence of tebentafusp is provided below and tebentafusp is further described in International Patent Publication No. WO 2011/001152 and U.S. Pat. Nos. 8,519,100 and 9,068,178, each of which is hereby incorporated by reference in its entirety. The alpha chain is composed of 195 amino acid residues and the beta chain is composed of 500 amino acid residues. The linker sequence between the two variable domains of the scFv antibody (24 amino acids) and the linker sequence between the scFv and TCR β chain variable region (5 amino acids) are underlined. The initiator methionine residues are post-translationally removed from both alpha and beta chains.

### Amino Acid Sequence of Tebentafusp-Tebn:

Alpha chain
(SEQ ID NO: 1)
AQQGEEDPQALSIQEGENATMNCSYKTSINNLQWYRQNSGRGLVHLILIR
SNEREKHSGRLRVTLDTSKKSSSLLITASRAADTASYFCATDGSTPMQFG
KGTRLSVIANIQKPDPAVYQLRDSKSSDKSVCLFTDFDSQTNVSQSKDSD
VYITDKC*VLDMRSMDFKSNSAVAWSNKSDFACANAFNNSIIPEDT
Anti-CD3 scFv Beta chain
(SEQ ID NO: 2)
AIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIKGGGGSGGGGSGGGGSGGGGSGGGSEVQLVESGGGLVQPGGSLR
LSCAASGYSFTGYTMNWVRQAPGKGLEWVALINPYKGVSTYNQKFKDRFT
CARSGYYGDSDWYFDVWGQGTLVTVSSGGGGSDGGITQSPKYLFRKEGQN
ISVDKSKNTAYLQMNSLRAEDTAVYYVTLSCEQNLNHDAMYWYRQDPGQG
LRLIYYSWAQGDFQKGDIAEGYSVSREKKESFPLTVTSAQKNPTAFYLCA
SSWGAPYEQYFGPGTRLTVTEDLKNVFPPEVAVFEPSEAEISHTQKATLV
CLATGFYPDHVELSWWVNGKEVHSGVC*TDPQPLKEQPALNDSRYALSSR
LRVSATFWQDPRNHFRCQVQFYGLSENDEWTQDRAKPVTQIVSAEAWGRA
D

### 6.2.2. IMC-F106C

In some embodiments, IMC-F106C is a T cell redirecting bispecific therapeutic agent comprising a soluble affinity enhanced TCR that binds to the SLLQHLIGL (SEQ ID NO:6) peptide-HLA-A*02 complex, fused to an anti-CD3 scFv. The targeting end of IMC-F106C (the soluble TCR) binds to a peptide fragment of the PRAME antigen (SLLQHLIGL (SEQ ID NO: 6) presented by HLA-A*02 on the surface of cancer cells.

IMC-F106C is composed of an alpha chain and a beta chain. The alpha chain comprises a TCR alpha chain variable region domain and a constant region domain. The beta chain comprises a TCR beta chain variable region domain and a constant region domainwhere the TCR beta chain is linked to a single-chain variable fragment (“scFv”) anti-CD3 antibody at its N-terminus. The linker sequence between the two variable domains of the scFv antibody (24 amino acids) and the linker sequence between the scFv and TCR β chain variable region (5 amino acids) are underlined. The two chains are covalently bonded via a disulfide bond between the cysteine at position 163 on the alpha chain amino acid sequence of SEQ ID NO:4 and the cysteine at position 429 on the anti-CD3 scFv beta chain amino acid sequence of SEQ ID NO:5. The amino acid sequence of IMC-F106C is provided below and IMC-F106C is described in International Patent Publication No. WO 2018/234319, which is incorporated herein by reference in its entirety.

### Amino Acid Sequence of IMC-F106C:

Alpha chain
(SEQ ID NO: 4)
GDAKTTQPNSMESNEEEPVHLPCNHSTISGTDYIHWYRQLPSQGPEYVIH
GLTSNVNNRMASLAIAEDRKSSTLILHRATLRDAAVYYCILILGHSRLGN
YIATFGKGTKLSVIPNIQNPDPAVYQLRDSKSSDKSVCLFTDFDSQTNVS
QSKDSDVYITDKCVLDMRSMDFKSNSAVAWSNKSDFACANAFNNSIIPED
T
Anti-CD3 scFv Beta chain
(SEQ ID NO: 5)
AIQMTQSPSSLSASVGDRVTITCRASQDIRNYLNWYQQKPGKAPKLLIYY
TSRLESGVPSRFSGSGSGTDYTLTISSLQPEDFATYYCQQGNTLPWTFGQ
GTKVEIKGGGGSGGGGSGGGGSGGGGSGGGSEVQLVESGGGLVQPGGSLR
LSCAASGYSFTGYTMNWVRQAPGKGLEWVALINPYKGVSTYNQKFKDRFT
ISVDKSKNTAYLQMNSLRAEDTAVYYCARSGYYGDSDWYFDVWGQGTLVT
VSSGGGGSDGGITQSPKYLFRKEGQNVTLSCEQNLNHDAMYWYRQDPGQG
LRLIYYSQIMGDEQKGDIAEGYSVSREKKESFPLTVTSAQKNPTAFYLCA
SSWWTGGASPIRFGPGTRLTVTEDLKNVFPPEVAVFEPSEAEISHTQKAT
LVCLATGFYPDHVELSWWVNGKEVHSGVCTDPQPLKEQPALNDSRYALSS
RLRVSATFWQDPRNHFRCQVQFYGLSENDEWTQDRAKPVTQIVSAEAWGR
AD

### 6.3. Circulating Tumor DNA (ctDNA)

In some embodiments of the present disclosure, circulating tumor DNA (ctDNA) is a subset of cell-free DNA (cfDNA) isolated from plasma or serum of a subject. In some embodiments, ctDNA originates from tumor cells. In some embodiments, ctDNA comprises fragments of tumor DNA.

In some embodiments, ctDNA is amplified by PCR and analyzed by next generation sequencing. In some embodiments, ctDNA is assessed for one or more mutations in one or more genes. In some embodiments, ctDNA is assessed for one or more mutations in GNAQ. In some embodiments, ctDNA is assessed for one or more mutations in GNA11. In some embodiments, ctDNA is assessed for one or more mutations in SF3B1. In some embodiments, ctDNA is assessed for one or more mutations in PLCB4. In some embodiments, ctDNA is assessed for one or more mutations in CYSLTR2. In some embodiments, ctDNA is assessed for one or more mutations in EIF1AX. In some embodiments, ctDNA is assessed for mutations in a panel of genes. In some embodiments, ctDNA is assessed for mutations in a panel of genes comprising GNAQ, GNA11, SF3B1, PLCB4, CYSLTR2, and EIF1AX.

### 6.4. Methods of Treatment

Disclosed herein are methods of treating cancer in a subject. In some embodiments, the method includes administering a T cell therapy to a subject who has or is suspected of having cancer. In some embodiments, the method includes administering tebentafusp to a subject who has or is suspected of having a gp100 positive cancer. In some embodiments, the method includes administering IMC-F106C to a subject who has or is suspected of having a PRAME positive cancer.

In some embodiments, the method of treating cancer includes measuring ctDNA levels in a subject.

### 6.4.1. Administration of T Cell Therapy

Disclosed herein are methods of administering a therapeutically effective amount of T cell therapy to a subject. In some embodiments, T cell therapy is administered by intravenous infusion or subcutaneous infusion. In some embodiments, tebentafusp is administered by direct injection into a tumor. In some embodiments, tebentafusp is administered by direct injection to a site close to tumor cells.

### 6.4.2. Dosing of Tebentafusp

Disclosed herein are methods of administering a therapeutically effective amount of tebentafusp to a subject. In some embodiments, the method includes administering tebentafusp at a dose ranging from about 10 mcg to about 68 mcg (e.g., about 10 mcg to about 60 mcg, about 10 mcg to about 55 mcg, about 10 mcg to about 50 mcg, about 10 mcg to about 45 mcg, about 10 mcg to about 40 mcg, about 10 mcg to about 35 mcg, about 10 mcg to about 30 mcg, about 10 mcg to about 25 mcg, about 10 mcg to about 20 mcg, about 10 mcg to about 15 mcg, about 15 mcg to about 68 mcg, about 15 mcg to about 60 mcg, about 15 mcg to about 55 mcg, about 15 mcg to about 50 mcg, about 15 mcg to about 45 mcg, about 15 mcg to about 40 mcg, about 15 mcg to about 35 mcg, about 15 mcg to about 30 mcg, about 15 mcg to about 25 mcg, about 15 mcg to about 20 mcg, about 20 mcg to about 68 mcg, about 20 mcg to about 60 mcg, about 20 mcg to about 55 mcg, about 20 mcg to about 50 mcg, about 20 mcg to about 45 mcg, about 20 mcg to about 40 mcg, about 20 mcg to about 35 mcg, about 20 mcg to about 30 mcg, about 20 mcg to about 25 mcg, about 25 mcg to about 68 mcg, about 25 mcg to about 60 mcg, about 25 mcg to about 55 mcg, about 25 mcg to about 50 mcg, about 25 mcg to about 45 mcg, about 25 mcg to about 40 mcg, about 25 mcg to about 35 mcg, about 25 mcg to about 30 mcg, about 30 mcg to about 68 mcg, about 30 mcg to about 60 mcg, about 30 mcg to about 55 mcg, about 30 mcg to about 50 mcg, about 30 mcg to about 45 mcg, about 30 mcg to about 40 mcg, about 30 mcg to about 35 mcg, about 35 mcg to about 68 mcg, about 35 mcg to about 60 mcg, about 35 mcg to about 55 mcg, about 35 mcg to about 50 mcg, about 35 mcg to about 45 mcg, about 35 mcg to about 40 mcg, about 40 mcg to about 68 mcg, about 40 mcg to about 60 mcg, about 40 mcg to about 55 mcg, about 40 mcg to about 50 mcg, about 40 mcg to about 45 mcg, about 45 mcg to about 68 mcg, about 45 mcg to about 60 mcg, about 45 mcg to about 55 mcg, about 45 mcg to about 50 mcg, about 50 mcg to about 68 mcg, about 50 mcg to about 60 mcg, about 50 mcg to about 55 mcg, about 55 mcg to about 68 mcg, about 55 mcg to about 60 mcg, or about 60 mcg to about 68 mcg).

In some embodiments, the method includes administering to a subject tebentafusp at a dose ranging from about 10 mcg to about 50 mcg (e.g., any of the values or subranges therein).

In some embodiments, the method includes administering to a subject tebentafusp at a dose ranging from about 68 mcg to about 200 mcg (e.g., about 68 mcg to about 180 mcg, about 68 mcg to about 160 mcg, about 68 mcg to about 140 mcg, about 68 mcg to about 120 mcg, about 68 mcg to about 100 mcg, about 68 mcg to about 80 mcg, about 80 mcg to about 200 mcg, about 80 mcg to about 180 mcg, about 80 mcg to about 160 mcg, about 80 mcg to about 140 mcg, about 80 mcg to about 120 mcg, about 80 mcg to about 100 mcg, about 100 mcg to about 200 mcg, about 100 mcg to about 180 mcg, about 100 mcg to about 160 mcg, about 100 mcg to about 140 mcg, about 100 mcg to about 120, about 120 mcg to about 200 mcg, about 120 mcg to about 180 mcg, about 120 mcg to about 160 mcg, about 120 mcg to about 140 mcg, about 140 mcg to about 200 mcg, about 140 mcg to about 180 mcg, about 140 mcg to about 160 mcg, about 160 mcg to about 200 mcg, about 160 mcg to about 180 mcg, or about 180 mcg to about 200 mcg).

In some embodiments, the method includes administering to a patient tebentafusp at a dose of about any one of 0.1 mcg, 0.5 mcg, 1.0 mcg, 1.5 mcg, 2.0 mcg, 2.5 mcg, 3.0 mcg, 3.5 mcg, 4.0 mcg, 4.5 mcg, 5.0 mcg, 5.5 mcg, 6.0 mcg, 6.5 mcg, 7.0 mcg, 7.5 mcg, 8.0 mcg, 9.0 mcg, 10 mcg, 11 mcg, 12 mcg, 13 mcg, 14 mcg, 15 mcg, 16 mcg, 17 mcg, 18 mcg, 19 mcg, 20 mcg, 21 mcg, 22 mcg, 23 mcg, 24 mcg, 25 mcg, 26 mcg, 27 mcg, 28 mcg, 29 mcg, 30 mcg, 31 mcg, 32 mcg, 33 mcg, 34 mcg, 35 mcg, 36 mcg, 37 mcg, 38 mcg, 39 mcg, 40 mcg, 41 mcg, 42 mcg, 43 mcg, 44 mcg, 45 mcg, 46 mcg, 47 mcg, 48 mcg, 49 mcg, 50 mcg, 51 mcg, 52 mcg, 53 mcg, 54 mcg, 55 mcg, 56 mcg, 57 mcg, 58 mcg, 59 mcg, 60 mcg, 61 mcg, 62 mcg, 63 mcg, 64 mcg, 65 mcg, 66 mcg, 67 mcg, 68 mcg, 69 mcg, 70 mcg, 71 mcg, 72 mcg, 73 mcg, 74 mcg, 75 mcg, 76 mcg, 77 mcg, 78 mcg, 79 mcg, 80 mcg, 81 mcg, 82 mcg, 83 mcg, 84 mcg, 85 mcg, 86 mcg, 87 mcg, 88 mcg, 89 mcg, 90 mcg, 91 mcg, 92 mcg, 93 mcg, 94 mcg, 95 mcg, 96 mcg, 97 mcg, 98 mcg, 99 mcg, 100 mcg, 105 mcg, 110 mcg, 115 mcg, 120 mcg, 125 mcg, 130 mcg, 135 mcg, 140 mcg, 145 mcg, 150 mcg, 155 mcg, 160 mcg, 165 mcg, 170 mcg, 175 mcg, 180 mcg, 185 mcg, 190 mcg, 195 mcg, 200 mcg or more.

In some embodiments, tebentafusp is administered in an intra-patient dose escalation regimen as described in U.S. Pat. Publication No. 2020/0040055A1, which is herein incorporated by reference in its entirety. In such embodiments, the intra-patient dose escalation regimen provides improved tolerability for treating gp100 positive tumors with a bi-specific therapeutic employing a T cell redirection mediated mechanism-of-action (i.e., tebentafusp). A non-limiting example of an intra-patient dose escalation for tebentafusp includes administering: at least one first dose of tebentafusp in the range of from 10-30 mcg; (b) at least one second dose of tebentafusp in the range of from 20-40 mcg, wherein the second dose is higher than the first dose; and then (c) at least one dose of at least 50 mcg of tebentafusp.

In some embodiments, tebentafusp is administered about once weekly. In some embodiments, tebentafusp is administered every 5 to 10 days, every 5 to 9 days, every 5 to 8 days, every 5 to 7 days, every 5 to 6 days, every 6 to 10 days, every 6 to 9 days, every 6 to 8 days, every 6 to 7 days, every 7 to 10 days, every 7 to 9 days, every 7 to 8 days, every 8 to 10 days, every 8 to 9 days, or every 9 to 10 days. In some embodiments, tebentafusp is administered every 7 days.

In some embodiments, tebentafusp is administered at a first dose of 20 mcg on Day 1, a second dose of 30 mcg on or around Day 8, a third dose of 68 mcg on or around Day 15, and a fourth dose of 68 mcg on or around Day 22.

In some embodiments, tebentafusp is administered once weekly in treatment cycles consisting of 4 weeks (28 days). In some embodiments, tebentafusp is administered for at least 1 cycle, at least 2 cycles, at least 3 cycles, at least 4 cycles, at least 5 cycles, at least 6 cycles, at least 7 cycles, at least 8 cycles, at least 9 cycles, or at least 10 cycles. In some embodiments, tebentafusp is administered for greater than 10 cycles, greater than 15 cycles, greater than 20 cycles, greater than 30 cycles, greater than 40 cycles, or greater than 50 cycles.

In some embodiments, tebentafusp is administered in multiple treatment cycles with at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, or at least 20 weeks between at least two treatment cycles.

In some embodiments, tebentafusp is administered in multiple treatment cycles without any off treatment weeks between treatment cycles.

Dosing of tebentafusp is further described in International Patent Publication No. WO 2017/208018 which is hereby incorporated by reference in its entirety.

### 6.4.3. Dosing of IMC-F106C

Disclosed herein are methods of administering a therapeutically effective amount of IMC-F106C to a subject. In some embodiments, the method includes administering IMC-F106C at a dose ranging from about 5 mcg to about 40 mcg (e.g., about 5 mcg to about 40 mcg, about 5 mcg to about 35 mcg, about 5 mcg to about 30 mcg, about 5 mcg to about 25 mcg, about 5 mcg to about 20 mcg, about 5 mcg to about 15 mcg, about 5 mcg to about 10 mcg, about 10 mcg to about 40 mcg, about 10 mcg to about 35 mcg, about 10 mcg to about 30 mcg, about 10 mcg to about 25 mcg, about 10 mcg to about 20 mcg, about 10 mcg to about 15 mcg, about 15 mcg to about 40 mcg, about 15 mcg to about 35 mcg, about 15 mcg to about 30 mcg, about 15 mcg to about 25 mcg, about 15 mcg to about 20 mcg).

In some embodiments, the method includes administering to a subject IMC-F106C at a dose ranging from about 15 mcg to about 80 mcg (e.g., about 15 mcg to about 75 mcg, about 15 mcg to about 70 mcg, about 15 mcg to about 65 mcg, about 15 mcg to about 60 mcg, about 15 mcg to about 55 mcg, about 15 mcg to about 50 mcg, about 15 mcg to about 45 mcg, about 15 mcg to about 40 mcg, about 15 mcg to about 35 mcg, about 15 mcg to about 30 mcg, about 15 mcg to about 25 mcg, about 15 mcg to about 20 mcg, about 20 mcg to about 75 mcg, about 20 mcg to about 70 mcg, about 20 mcg to about 65 mcg, about 20 mcg to about 60 mcg, about 20 mcg to about 55 mcg, about 20 mcg to about 50 mcg, about 20 mcg to about 45 mcg, about 20 mcg to about 40 mcg, about 20 mcg to about 35 mcg, about 20 mcg to about 30 mcg, about 20 mcg to about 25 mcg, about 25 mcg to about 75 mcg, about 25 mcg to about 70 mcg, about 25 mcg to about 65 mcg, about 25 mcg to about 60 mcg, about 25 mcg to about 55 mcg, about 25 mcg to about 50 mcg, about 25 mcg to about 45 mcg, about 25 mcg to about 40 mcg, about 25 mcg to about 35 mcg, about 25 mcg to about 30 mcg, about 30 mcg to about 75 mcg, about 30 mcg to about 70 mcg, about 30 mcg to about 65 mcg, about 30 mcg to about 60 mcg, about 30 mcg to about 55 mcg, about 30 mcg to about 50 mcg, about 30 mcg to about 45 mcg, about 30 mcg to about 40 mcg, about 30 mcg to about 35 mcg, about 30 mcg to about 40 mcg, about 35 mcg to about 75 mcg, about 35 mcg to about 70 mcg, about 35 mcg to about 65 mcg, about 35 mcg to about 60 mcg, about 35 mcg to about 55 mcg, about 35 mcg to about 50 mcg, about 35 mcg to about 45 mcg, about 40 mcg to about 75 mcg, about 40 mcg to about 70 mcg, about 40 mcg to about 65 mcg, about 40 mcg to about 60 mcg, about 40 mcg to about 55 mcg, about 40 mcg to about 50 mcg, about 40 mcg to about 45 mcg, about 45 mcg to about 75 mcg, about 45 mcg to about 70 mcg, about 45 mcg to about 65 mcg, about 45 mcg to about 60 mcg, about 45 mcg to about 55 mcg, about 45 mcg to about 50 mcg, about 50 mcg to about 75 mcg, about 50 mcg to about 70 mcg, about 50 mcg to about 65 mcg, about 50 mcg to about 60 mcg, about 50 mcg to about 55 mcg, about 55 mcg to about 75 mcg, about 55 mcg to about 70 mcg, about 55 mcg to about 65 mcg, about 55 mcg to about 60 mcg, about 60 mcg to about 75 mcg, about 60 mcg to about 70 mcg, about 60 mcg to about 65 mcg, about 65 mcg to about 75 mcg, about 65 mcg to about 70 mcg, about 70 mcg to about 75 mcg, or about 75 mcg to about 80 mcg).

In some embodiments, the method includes administering to a patient IMC-F106C at a dose of about any one of 0.1 mcg, 0.5 mcg, 1.0 mcg, 1.5 mcg, 2.0 mcg, 2.5 mcg, 3.0 mcg, 3.5 mcg, 4.0 mcg, 4.5 mcg, 5.0 mcg, 5.5 mcg, 6.0 mcg, 6.5 mcg, 7.0 mcg, 7.5 mcg, 8.0 mcg, 9.0 mcg, 10 mcg, 11 mcg, 12 mcg, 13 mcg, 14 mcg, 15 mcg, 16 mcg, 17 mcg, 18 mcg, 19 mcg, 20 mcg, 21 mcg, 22 mcg, 23 mcg, 24 mcg, 25 mcg, 26 mcg, 27 mcg, 28 mcg, 29 mcg, 30 mcg, 31 mcg, 32 mcg, 33 mcg, 34 mcg, 35 mcg, 36 mcg, 37 mcg, 38 mcg, 39 mcg, 40 mcg, 41 mcg, 42 mcg, 43 mcg, 44 mcg, 45 mcg, 46 mcg, 47 mcg, 48 mcg, 49 mcg, 50 mcg, 51 mcg, 52 mcg, 53 mcg, 54 mcg, 55 mcg, 56 mcg, 57 mcg, 58 mcg, 59 mcg, 60 mcg, 61 mcg, 62 mcg, 63 mcg, 64 mcg, 65 mcg, 66 mcg, 67 mcg, 68 mcg, 69 mcg, 70 mcg, 71 mcg, 72 mcg, 73 mcg, 74 mcg, 75 mcg, 76 mcg, 77 mcg, 78 mcg, 79 mcg, 80 mcg, 81 mcg, 82 mcg, 83 mcg, 84 mcg, 85 mcg, 86 mcg, 87 mcg, 88 mcg, 89 mcg, 90 mcg, 91 mcg, 92 mcg, 93 mcg, 94 mcg, 95 mcg, 96 mcg, 97 mcg, 98 mcg, 99 mcg, 100 mcg, 105 mcg, 110 mcg, 115 mcg, 120 mcg, 125 mcg, 130 mcg, 135 mcg, 140 mcg, 145 mcg, 150 mcg, 155 mcg, 160 mcg, 165 mcg, 170 mcg, 175 mcg, 180 mcg, 185 mcg, 190 mcg, 195 mcg, 200 mcg, 210 mcg, 220 mcg, 230 mcg, 240 mcg, 250 mcg, 260 mcg, 270 mcg, 280 mcg, 290 mcg, 300 mcg, 310 mcg, 320 mcg, 330 mcg, 340 mcg, 350 mcg, 360 mcg, 370 mcg, 380 mcg, 390 mcg, 400 mcg, or more.

In some embodiments, IMC-F106C is administered in an intra-patient dose escalation regimen. A non-limiting example of an intra-patient dose escalation for and (c) at least one dose of at least 50 mcg of IMC-F106C includes administering: at least one first dose of IMC-F106C in the range of from 5 to 40 mcg; (b) at least one second dose of IMC-F106C in the range of from 15 to 80 mcg; and (c) at least one third dose of IMC-F106C in the range of from 60 to 400 mcg, wherein the second dose is higher than the first dose and the third dose is higher than the second dose.

In some embodiments, IMC-F106C is administered about once weekly. In some embodiments, IMC-F106C is administered every 5 to 10 days, every 5 to 9 days, every 5 to 8 days, every 5 to 7 days, every 5 to 6 days, every 6 to 10 days, every 6 to 9 days, every 6 to 8 days, every 6 to 7 days, every 7 to 10 days, every 7 to 9 days, every 7 to 8 days, every 8 to 10 days, every 8 to 9 days, or every 9 to 10 days. In some embodiments, IMC-F106C is administered every 7 days.

In some embodiments, IMC-F106C is administered at a first dose of 6 mcg on Day 1, a second dose of 20 mcg on or around Day 8, and a third dose of 80 mcg on or around Day 15.

In some embodiments, IMC-F106C is administered at a first dose of 15 mcg on Day 1, a second dose of 40 mcg on or around Day 8, and a third dose of 160 mcg on or around Day 15.

In some embodiments, IMC-F106C is administered at a first dose of 20 mcg on Day 1, a second dose of 60 mcg on or around Day 8, and a third dose of 240 mcg on or around Day 15.

In some embodiments, IMC-F106C is administered at a first dose of 20 mcg on Day 1, a second dose of 60 mcg on or around Day 8, and a third dose of 160 mcg on or around Day 15.

In some embodiments, IMC-F106C is administered at a first dose of 20 mcg on Day 1, a second dose of 60 mcg on or around Day 8, and a third dose of 320 mcg on or around Day 15.

In some embodiments, IMC-F106C is administered at a first dose of 20 mcg on Day 1, a second dose of 60 mcg on or around Day 8, and a third dose of 160 mcg, 200 mcg, 240 mcg, 280 mcg, or 320 mcg on or around Day 15.

In some embodiments, IMC-F106C is administered once weekly in treatment cycles consisting of 3 weeks (21 days). In some embodiments, IMC-F106C is administered for at least 1 cycle, at least 2 cycles, at least 3 cycles, at least 4 cycles, at least 5 cycles, at least 6 cycles, at least 7 cycles, at least 8 cycles, at least 9 cycles, or at least 10 cycles. In some embodiments, IMC-F106C is administered for greater than 10 cycles, greater than 15 cycles, greater than 20 cycles, greater than 30 cycles, greater than 40 cycles, or greater than 50 cycles.

In some embodiments, IMC-F106C is administered in multiple treatment cycles with at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, or at least 20 weeks between at least two treatment cycles.

In some embodiments, IMC-F106C is administered in multiple treatment cycles without any off treatment weeks between treatment cycles.

Dosing of IMC-F106C is further described in U.S. Patent Application No. 63/285,044, filed Dec. 1, 2021 and U.S. patent application Ser. No. ______, filed Aug. 31, 2022, both of which are entitled “Treatment of PRAME positive cancer” and are hereby incorporated by reference in their entireties.

### 6.4.4. Diseases and Disorders

In some embodiments, T cell therapy is used for treating a disease or disorder. In some embodiments, the disease or disorder is cancer. In some embodiments, the T cell therapy is characterized by the presence of lesions or tumors in a patient. In some embodiments the lesions or tumors express gp100. In some embodiments the lesions or tumors express PRAME.

In some embodiments, T cell therapy is used for treating cancer. In some embodiments, the cancer to be treated is selected from melanoma, breast cancer, lung cancer, endometrial cancer, and ovarian cancer.

In some embodiments, T cell therapy is used for treating melanoma. In some embodiments, the melanoma is cutaneous melanoma, uveal melanoma, superficial spreading melanoma, lentigo maligna melanoma, nodular melanoma, mucosal melanoma, polypoid melanoma, desmoplastic melanoma, amelanotic melanoma, soft-tissue melanoma, or acral ientiginous melanoma. In some embodiments, the melanoma is at any of stage I, II, III, or IV, according to the American Joint Committee on Cancer (AJCC) staging groups. In some embodiments, the melanoma is advanced. In some embodiments, the melanoma is metastatic. In some embodiments, the melanoma is recurrent. In some embodiments, the melanoma is refractory (e.g., resistant to treatment).

In some embodiments, the cancer to be treated has an oncogenic mutation. In some embodiments, the cancer has a mutation in at least one of GNAQ, GNA11, PLCB4, CYSLTR2, EIFAIX, BAP1, and SF3B1.

### 6.4.5. Clinical Assessment

In some embodiments, the overall survival rate at 1 year following initial administration of T cell therapy is at least about 60%. In some embodiments, the overall survival rate at 1 year is about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%. In some embodiments, the overall survival rate at 1 year is greater than about 90%.

In some embodiments of the disclosure, patients have stable disease (SD) according to RECIST v1.1 and/or irRECIST criteria following four (4) or more weeks of weekly treatment with T cell therapy. In some embodiments, patients have stable disease (SD) according to RECIST v1.1 and/or irRECIST criteria following at least 5 weeks of weekly treatment with T cell therapy. In some embodiments, patients have stable disease (SD) according to RECIST v1.1 and/or irRECIST criteria following weekly treatment with T cell therapy for 6, 7, 8, 9, 10, 11, 12, 13, 14, 15, 16, 17, 18, 19, 20, 21, 22, 23, or 24 weeks. In some embodiments, patients have stable disease (SD) according to RECIST v1.1 and/or irRECIST criteria following weekly treatment with T cell therapy for greater than 24 weeks.

In some embodiments of the disclosure, patients have progressive disease (PD) according to RECIST v1.1 and/or irRECIST criteria following four (4) or more weeks of weekly treatment with T cell therapy. In some embodiments, patients have progressive disease (PD) according to RECIST v1.1 and/or irRECIST criteria following at least 5 weeks of weekly treatment with T cell therapy. In some embodiments, patients have progressive disease (PD) according to RECIST v1.1 and/or irRECIST criteria following weekly treatment with T cell therapy for 6, 7, 8, 9, 10, 11, 12, 13, 14, 15, 16, 17, 18, 19, 20, 21, 22, 23, or 24 weeks. In some embodiments, patients have progressive disease (PD) according to RECIST v1.1 and/or irRECIST criteria following weekly treatment with T cell therapy for greater than 24 weeks.

In some embodiments of the disclosure, patients achieve a partial response (PR) according to RECIST v1.1 and/or irRECIST criteria following four (4) or more weeks of weekly treatment with T cell therapy. In some embodiments, patients achieve a partial response (PR) according to RECIST v1.1 and/or irRECIST criteria following at least 5 weeks of weekly treatment with T cell therapy. In some embodiments, patients achieve a partial response (PR) according to RECIST v1.1 and/or irRECIST criteria following weekly treatment with T cell therapy for 6, 7, 8, 9, 10, 11, 12, 13, 14, 15, 16, 17, 18, 19, 20, 21, 22, 23, or 24 weeks. In some embodiments, patients achieve a partial response (PR) according to RECIST v1.1 and/or irRECIST criteria following weekly treatment with T cell therapy for greater than 24 weeks.

In some embodiments of the disclosure, patients treated with T cell therapy experience progression-free survival (PFS) at least about one (1) month. In some embodiments, patients treated with T cell therapy experience PFS at least about 1.5 months. In some embodiments, patients treated with T cell therapy experience PFS at least about 2 months, 2.2 months, 2.4 months, 2.6 months, 2.8 months, 3 months, 3.2 months, 3.4 months, 3.6 months, 3.8 months, 4 months, 4.2 months, 4.4 months, 4.6 months, 4.8 months, 5 months, 5.2 months, 5.4 months, 5.6 months, 5.8 months, or 6 months. In some embodiments, patients treated with tebentafusp experience PFS greater than 6 months, 7 months, 8 months, 9 months, 10 months, 11 months, 12 months, 13 months, 14 months, 15 months, 16 months or 18 months.

In some embodiments, patients treated with T cell therapy experience tumor shrinkage. In some embodiments, the change in tumor size from baseline is a decrease of at least about 1%, 5%, 10%, 15%, 20%, 25%, 30%, 35%, 40%, 45%, 50%, 55%, 60%, 65%, 70%, 75%, 80%, 85%, 90%, 95%, 99%, or greater than 99%.

In some embodiments, patients treated with T cell therapy experience tumor growth. In some embodiments, the change in tumor size from baseline is an increase of at least about 1%, 5%, 10%, 15%, 20%, 25%, 30%, 35%, 40%, 45%, 50%, 55%, 60%, 65%, 70%, 75%, 80%, 85%, or 90%.

### 6.5. Patient Population

In some embodiments, the patient is positive for a human leukocyte antigen serotype of HLA-A*02. In some embodiments, the patient has a YLEPGPVTA (SEQ ID NO: 3) peptide-HLA-A*02 complex. In some embodiments, the patient has a SLLQHLIGL (SEQ ID NO: 6) peptide-HLA-A*02 complex.

In some embodiments, the patient has or is suspected of having melanoma. In some embodiments, the patient has or is suspected of having advanced melanoma. In some embodiments, the patient has or is suspected of having metastatic melanoma.

In some embodiments, the patient has or is suspected of having cancer selected from breast cancer, endometrial cancer, ovarian cancer, and lung cancer.

In some embodiments, the patient has cancer that has progressed following treatment for the cancer (e.g., refractory or resistant cancer). In some embodiments, the patient has cancer that has progressed following treatment with radiation or chemotherapy. In some embodiments, the patient has cancer that has progressed following treatment with an immune checkpoint inhibitor (e.g., anti-CLA-4 agents (e.g., ipilimumab) and anti-PD (L) 1 agents (e.g., nivolumab).

### 7. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### 7.1. Methods

### 7.1.1. Outcome Measures

Treatment efficacy was assessed using Response Evaluation Criteria in Solid Tumors (RECIST v1.1) and Kaplan-Meier survival analysis. Overall survival (OS) was measured from the start of treatment to time of death. Patients were censored on the last date they were known to be alive.

Adverse events were assessed by the investigator and graded per the National Cancer Institute Common Terminology Criteria for Adverse Events (CTCAE), version 4.03, except for cytokine release syndrome which was graded according to 2019 ASTCT Consensus Grading for Cytokine Release Syndrome (Lee, et al., Biol Blood Marrow Tr 25, 625-638 (2019)).

Rash is a composite term for a list of skin toxicities of any grade.

### 7.1.2. ctDNA Analysis

Serum samples, collected at baseline and at weeks 5, 9, and 25 on-treatment were used to assess ctDNA level of circulating tumor DNA. The analysis focused on mutations up to and including the week 9 on-treatment timepoint since patient data was sparse at the week 25 timepoint. A custom panel of mutations commonly found in uveal melanoma was designed to assess changes in ctDNA (Table 1). ctDNA was amplified by multiplex PCR and analyzed by next-generation sequencing. Variants with allele frequencies≤0.3% at baseline were excluded from the analysis.

Survival analysis was carried out using R package survminer v0.4.8 and Cox likelihood ratio test was used to assess differences between the survival curves. Univariate Cox proportional-hazards methods were used to model the prognostic importance of potential predictors of survival.

TABLE 1
Uveal specific mutations for ctDNA mutation profiling

| Gene | HGVSp |
|---|---|
| GNAQ | p.Q209L |
| | p.Q209P |
| GNA11 | p.Q209L |
| SF3B1 | p.K700E |
| | p.R625L |
| | p.R625H |
| | p.R625C |
| PLCB4 | p.D630Y |
| | p.D630V |
| CYSLTR2 | p.L129Q |

### 7.1.3. Tumor Mutation Analysis

Tumor biopsies were analyzed for mutations in GNAQ, GNA11, PLCB4, CYSLTR2, SF3B1 and EIF1AX. DNA libraries were generated from tumor biopsy samples, which were snap frozen, using the Illumina ExomeSeq all exon v6 kit. Paired end fragments of 100 bp length were sequenced (50 million reads per sample) using the Illumina NovaSeq system. The resulting reads were aligned using BWA-MEM (version 0.7.15). Reads were mapped to the GRCh38 primary assembly provided by Ensembl. Duplicate reads were flagged using the MarkDuplicate function of Picard to prevent variant call errors. Somatic variants were called using MuTect2 (GATK Somatic SNVs and INDELs 4.1.6.0).

### 7.1.4. Statistical Analysis

Approximately 150 patients were to be enrolled, including a minimum of 120 patients for RECIST v1.1 evaluation. With 120 patients and an observed objective response rate (ORR) of 10% or more, the precision around estimation of ORR was assessed to be 5.3% to 16.8% by 95% confidence intervals. The primary analysis of the study was conducted after ≥120 evaluable patients had been enrolled and followed for ≥12 months from the start of treatment.

Data were analyzed and reported based on all patient data up to the data cut-off date of Mar. 20, 2020, by which time all patients had at least one year of follow-up from the start of treatment. Confidence intervals for ORR and related endpoints were calculated using exact methods. Time-to-event endpoints such as overall survival (OS) were analyzed graphically through Kaplan-Meier methods and the median and 95% confidence intervals calculated by the method of Brookmeyer and Crowley.

The analysis set presented includes all patients with measurable disease at baseline according to RECIST v1.1, who received at least one full or partial dose of tebentafusp (n=127). All safety analyses were performed using the Safety Analysis Set (SAF), which includes all patients who received at least one (1) full or partial dose of tebentafusp (n=127).

Subgroup analyses of best overall response (BOR), OS, and progression-free survival (PFS) were conducted for a number of covariates relating to line of therapy, prior therapy, best response to prior therapy, prior IO checkpoint inhibitors and prior immunotherapy

### 7.2. Example 1: Phase 1/2 Study of Tebentafusp in Subjects with Metastatic Uveal Melanoma (mUM)

### 7.2.1. Study Description and Design

This open-label, international, phase 1/2 study included a phase 1 dose-escalation as well as an expansion cohort and was subsequently expanded into a full phase 2 study. Results of the phase 1 dose-escalation portion demonstrated that a step-up dosing regimen of tebentafusp permitted a 36% increase in the recommended phase 2 dose (RP2D) compared with weekly fixed dosing, with efficacy shown in treatment of metastatic uveal melanoma with minimal side effects (Carvajal, R. D., et al., J Clin Onc 17, 1939-1948 (2022)).

The primary objectives of the phase 2 portion were to assess safety and to estimate the objective response rate (ORR) based on RECIST v1.1 (Eisenhauer, E. A., et al., Eur J Cancer 45, 228-247 (2009)) in patients treated at the recommended phase 2 dose (RP2D) of tebentafusp. Secondary objectives included assessment of the antitumor efficacy of tebentafusp with the parameters of overall survival (OS), progression-free survival (PFS), disease control rate (DCR), time to response, duration of response (DOR), and the rate and duration of minor response (MR; defined as tumor response with a 10%-29% reduction in the sum of the longest diameters (SLD)).

Patients in the phase 2 study received weekly intravenous tebentafusp, initially at 20 mcg at week 1 and 30 mcg at week 2, then at the RP2D of 68 mcg starting at week 3 and beyond. Patients with an initial assessment of progressive disease (PD) according to RECIST v1.1 were allowed to continue therapy beyond initial progressive disease, provided that they did not have symptomatic progression requiring alternative therapy and provided that the investigator believed they were continuing to derive clinical benefit. The modified immune-related RECIST (irRECIST) criteria (Bohnsack, O., et al., J Clin Oncol 32, e22121 (2014)) was used to evaluate response to treatment beyond progression. Treatment post RECIST-PD was continued until confirmed immune-related progressive disease (irPD) defined as an additional ≥20% increase in tumor burden (determined by the sum of diameters of both target and new measurable lesions) per modified irRECIST criteria.

Tumor-based endpoints were assessed by a blinded independent central review (BICR) with investigator assessment data collected as a secondary evaluation. To assess potential predictors of efficacy of tebentafusp, change in serum ctDNA level in response to treatment was also measured.

### 7.2.2. Study Population

Eligibility criteria for enrollment in the study included being greater than or equal to 18 years of age and having a histologically or cytologically confirmed diagnosis of metastatic uveal melanoma (mUM), a life expectancy of greater than 3 months as estimated by the investigator, a positive test for HLA-A*02:01 as assessed by central assay, measurable disease according to RECIST v1.1, experience of disease progression while on one or two prior lines of therapy (chemotherapy, immunotherapy, or targeted therapy) in the metastatic or advanced setting, and an Eastern Cooperative Oncology Group (ECOG) performance score of ≤1. Patients were excluded from the study if they had symptomatic or untreated central nervous system (CNS) metastases or CNS metastases that required doses of corticosteroids within three weeks prior to Study Day 1, a history of severe hypersensitivity reactions to other biologic drugs or monoclonal antibodies, out-of-range protocol defined laboratory parameters, or clinically significant cardiac disease or impaired cardiac function.

Of the 148 HLA-A*02:01 positive patients screened, 127 patients met the eligibility criteria and were enrolled between January and December 2017 across 26 centers in five countries (Canada, Germany, Spain, United Kingdom, and United States). The median age was 61 years (range, 25-88 years), and approximately half were male.

The majority of patients (96%) had hepatic involvement. A total of 53% of patients had either American Joint Committee on Cancer (AJCC) Mlb or Mlc disease and 58% of patients had a baseline LDH above the upper limit of normal (ULN). The median time from initial diagnosis to the development of metastatic disease was 3 years (range, 0-28). All patients had received at least one prior line of therapy in the metastatic setting, with 34% receiving ≥2 lines of prior systemic (± liver-directed) therapy. More than two-thirds of patients (n=90) received prior immune checkpoint inhibition, of whom 68% had primary resistance to treatment with a prior best response of PD, and 32% relapsed, with a prior best response of at least SD, following treatment. At the time of data cut-off, the median follow-up was 19.5 months (95% CI: 16-22.2 months).

Analysis of tumour biopsies (n=63) revealed 23 (37%) had mutations in the gene encoding GNAQ, 26 (41%) in GNA11, 3 (5%) in CYSLTR2, 1 (2%) in PLCB4 and 11 (17%) in SF3B1.

All 127 (100%) patients received study drug, of whom 21 (17%) remained on treatment at the time of data cut-off. The median duration of treatment was 5.5 months (range 0-35). The primary reason for treatment discontinuation was disease progression (70%); 6 (5%) patients discontinued treatment due to an adverse event (AE) regardless of causality. At the data cut-off date, 53 (42%) patients remained on study and 74 (58%) patients were reported to have ended the study. Death was the primary cause of study discontinuation (69/74; 93%). Nearly all deaths (67/69) were related to disease progression; the cause of death in 2 patients was listed as ‘other’, including cerebrovascular event in the setting of a fall in one, and clinical disease progression in the other. There were no deaths due to adverse events or caused by the study drug.

### 7.2.3. Study Treatment and Administration

Tebentafusp was administered intravenously to patients in the phase 2 portion of the study at a dose of 20 mcg on cycle 1 day 1 (C1D1), 30 mcg on cycle 1 day 8, (C1D8), and then the recommended phase 2 dose (RP2D) of 68 mcg on cycle 1 day 15 (C1D15) and weekly (QW) thereafter.

### 7.2.4. Study Assessments and Procedures

Following at least the first three infusions, patients were observed for a minimum of sixteen hours for vital signs monitoring and, if necessary, provision of supportive care. After this induction period and provided no Grade 2 or higher hypotension was noted, the observation period following subsequent infusions of tebentafusp could be reduced to 30 to 60 minutes.

Treatment continued until confirmed disease progression as per modified immune-related RECIST (irRECIST) criteria, intolerable toxicity, investigator decision, or patient withdrawal of consent. The modification to irRECIST was to redefine confirmed immune-resistant progressive disease (irPD) as unequivocal progression of non-target lesions and/or new non-measurable disease or an additional 20% increase in tumor burden (as determined by the sum of diameters of both target and measurable new lesions) from initial PD assessment per RECISTv1.1 rather than from the nadir.

An independent data monitoring committee (IDMC) was established to provide oversight of safety and efficacy considerations and give advice and recommendations regarding steps to ensure both patient safety and the ethical integrity of the study. Radiologic assessments were performed every eight weeks from Cycle 1 Day 1 (C1D1) until C11D1 (40 weeks), then every 12 weeks until PD per RECIST v1.1, irPD per modified irRECIST for pateints who continued treatment beyond PD per RECIST v1.1 or discontinuation of study drug.

### 7.2.5. Results

Data from this study provide the longest follow-up of overall survival (OS) and safety of a soluble TCR therapeutic to date. The observed 1-year OS rate was 62% and median OS was 16.8 months.

### 7.2.5.1 Safety and Adverse Events

All patients experienced at least one treatment-related adverse event (TRAE) (Table 2). The most frequently reported TRAEs of any grade could be classified as skin related, due to targeting of gp100+ melanocytes, or cytokine related, due to T cell activation, and included rash (87%), pyrexia (80%), pruritus (67%), and chills (64%). 51 (40%) patients had a Grade 3 event as their maximum grade, a third of which were rash events (n=20) and 8 (6%) patients experienced a Grade 4 related AE (hypotension and multiple organ dysfunction syndrome in one patient, lymphopenia, γ-glutamyltransferase increased, atrial fibrillation, amylase increased, hypophosphatemia, hypokalemia, and aspartate aminotransferase increased). A total of 25 (20%), 21 (17%) and 4 (3%) patients experienced TRAEs leading to hospitalization, dose interruptions and drug discontinuation, respectively. Following the 16-hour observation period after the first 3 doses, 7 (6%) patients required an additional overnight stay due to a treatment-related AE. TRAEs leading to discontinuation included atrial fibrillation and cytokine release syndrome (CRS), multiple organ dysfunction syndrome and CRS, left ventricular dysfunction, and dyspnoea. There were no treatment-related deaths.

TABLE 2
Most common treatment-related adverse events (AEs)

| | | |
|---|---|---|
| AE, related | Any grade N (%) | Grade ≥ 3 (%) N (%) |

| Any AE | 127 | (100) | 59 | (46.5) |
|---|---|---|---|---|
| Cytokine-mediated | | | | |
| CRS^{a} | 109 | (86) | 5 | (4) |
| Pyrexia | 101 | (80) | 5 | (4) |
| Chills | 81 | (64) | 1 | (1) |
| Nausea | 75 | (59) | 2 | (2) |
| Fatigue | 66 | (52) | 4 | (3) |
| Hypotension | 52 | (41) | 10 | (8) |
| Vomiting | 44 | (35) | 1 | (1) |
| Headache | 30 | (24) | 1 | (1) |
| Skin-related | | | | |
| Rash^{b} | 111 | (87) | 20 | (16) |
| Pruritus | 85 | (67) | 5 | (4) |
| Dry skin | 50 | (39) | 1 | (1) |
| Periorbital edema | 34 | (27) | 0 | |
| Edema peripheral | 33 | (26) | 1 | (1) |

| | | | |
|---|---|---|---|
| Hair color changes | 32 | (25) | 0 |
| Skin exfoliation | 28 | (22) | 0 |

Table summarizes treatment related AEs that were present at least 20% any grade
^{a}Cytokine release syndrome (CRS) was graded according to 2019 ASTCT Consensus Grading for CRS
^{b}Rash is a composite term for a list of skin toxicities of any grade

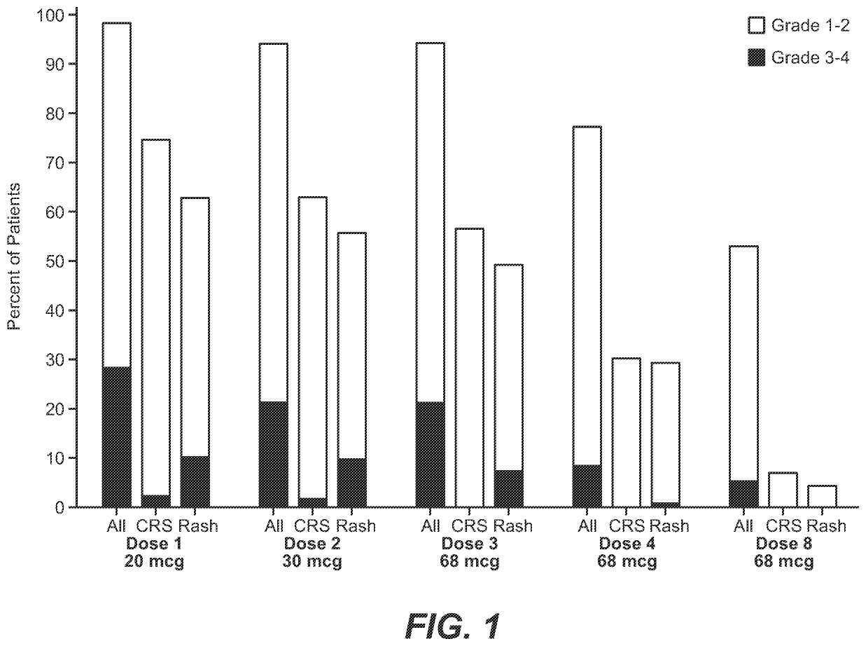

Consistent with the Phase 1 studies, adverse events related to tebentafusp, including rash, generally occurred early on-treatment and reduced in incidence and severity with repeated dosing, with ~65% of patients experiencing rash from weeks 1 to 3 and 23% in Week 8 (FIG. 1). Patients with symptomatic rash were generally managed successfully with antihistamine and topical corticosteroid therapy, and no patient discontinued treatment due to rash.

CRS is a common adverse event with T cell engaging therapies and 109 (86%) patients experienced CRS based on American Society for Transplantation and Cellular Therapy (ASTCT) consensus grading criteria (Lee, D W, et al., Biol Blood Marrow Tr 25, 625-638 (2019)). Most patients had either grade 1 (33%) or grade 2 (49%) CRS as their maximum grade. Very few patients experienced grade 3 (3.1%) or 4 (0.8%) events; however, 2 CRS events did lead to treatment discontinuation (one grade 3 and one grade 4). The onset of CRS, based on increase in body temperature, generally began within 8-10 hours of administration and, as with other TRAEs, most events occurred following the first 3 doses, with a marked reduction in the incidence and severity of CRS thereafter (FIG. 1). All five grade 3 and 4 episodes occurred following one of the initial 2 doses, during the step-up dosing regimen. Patients were generally treated with antipyretics (n=96, 88%), intravenous fluids (n=48, 44%) and/or systemic glucocorticoids (n=28, 26%). Supplemental oxygen (n=9, 8%), vasopressors (n=2, 2%) and tocilizumab (n=2, 2%) were less frequently used to manage more severe cases.

### 7.2.5.2 Tumor Response and Progression-Free Survival

The primary endpoint of ORR per RECIST v1.1 by independent central review was 5% (95% CI: 2-10%) (Table 3), with 6 patients achieving a partial response (PR). Among these 6 patients, 3 had ongoing responses for >6 months, 1 had an ongoing response for >12 months and 1 patient was censored at 5.3 months. The median DOR was 8.7 months (95% CI: 5.6-24.5) and the median time to response was 4.6 months, with response times ranging from 1.6 to 20.5 months.

TABLE 3
Best overall RECIST response rate

| | Tebentafusp | |
|---|---|---|
| | (n = 127) | |
| | No. (%) | |
| Objective response rate | 6 | (5%) |
| Partial response | 6 | (5%) |
| Stable disease^{a} | 57 | (45%) |
| Minor response^{b} | 8 | (6%) |
| Progressive Disease | 60 | (47%) |
| Non-Evaluable/Not applicable | 4 | (3%) |
| Disease control rate-24 w (CR/PR/SD) [1] | 29 | (23%) |

Tumour assessment was based on RECIST v1.1 by Independent Central Review.
^{a}Stable disease ≥ 8 weeks
^{b}A minor response was defined as a reduction from baseline in the sum of longest diameters (or short axis for lymph nodes) of target lesions (mm) of 10% to 29%, where non-target lesion response was not unequivocal progression, and no new lesions were present. Confirmation was required after ≥ 4 weeks.

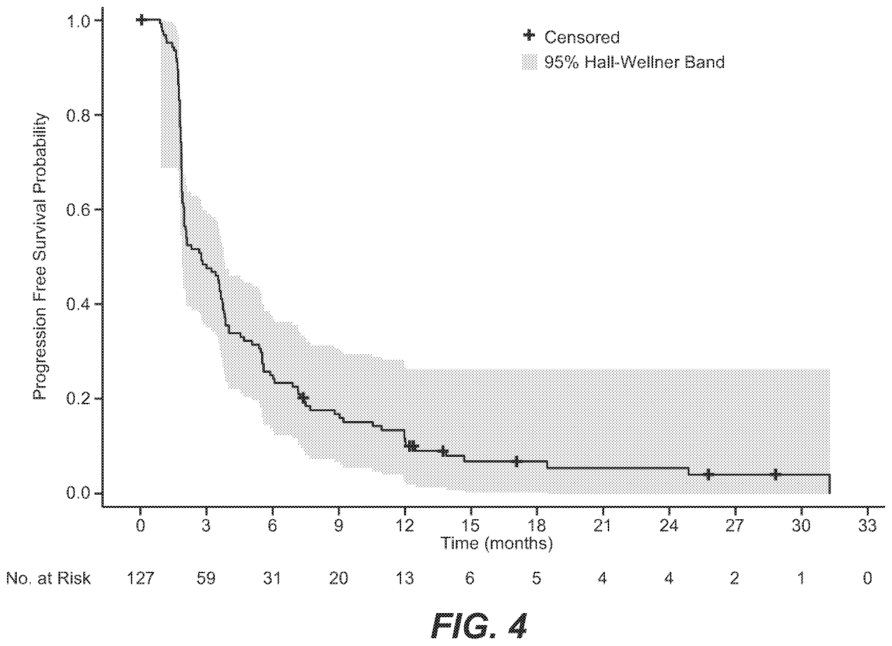

Of the 127 patients, 57 (45%) achieved SD at ≥8 weeks. The DCR was 32% (n=40; 95% CI: 24-40%) at ≥16 weeks and 23% (n=29; 95% CI: 16-31%) at ≥24 weeks. Any tumour shrinkage of target lesions was observed in 44% (n=51) of evaluable patients (n=116), including 10 of 55 (18%) patients with a best RECIST response of PD (FIG. 4), consistent with radiographic pseudoprogression.

The median progression-free survival (PFS) was 2.8 months (95% CI: 2-3.6). The estimated PFS rates were 25% (95% CI: 18-33%) at 6 months and 11% (95% CI: 6-17%) at 12 months (FIG. 4). A total of 90 patients (71%) were treated beyond initial disease progression, with a median duration of treatment following RECIST-PD of 2.9 months (range: 0-23.1 months). Of these patients, 6 (7%) achieved irSD, and 69 (77%) had confirmed irPD, per modified irRECIST as assessed by blinded independent central review.

### 7.2.5.3 Overall Survival

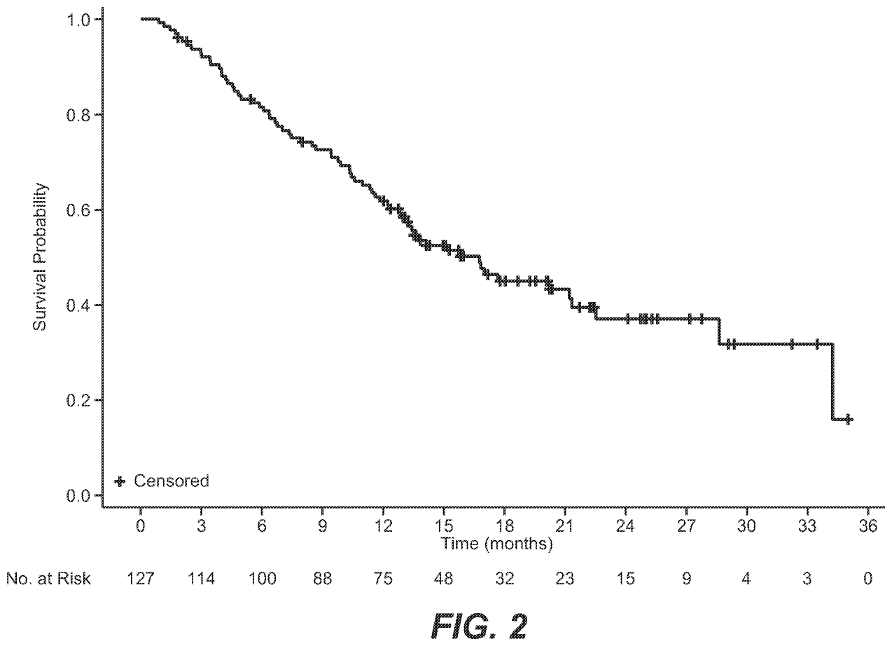

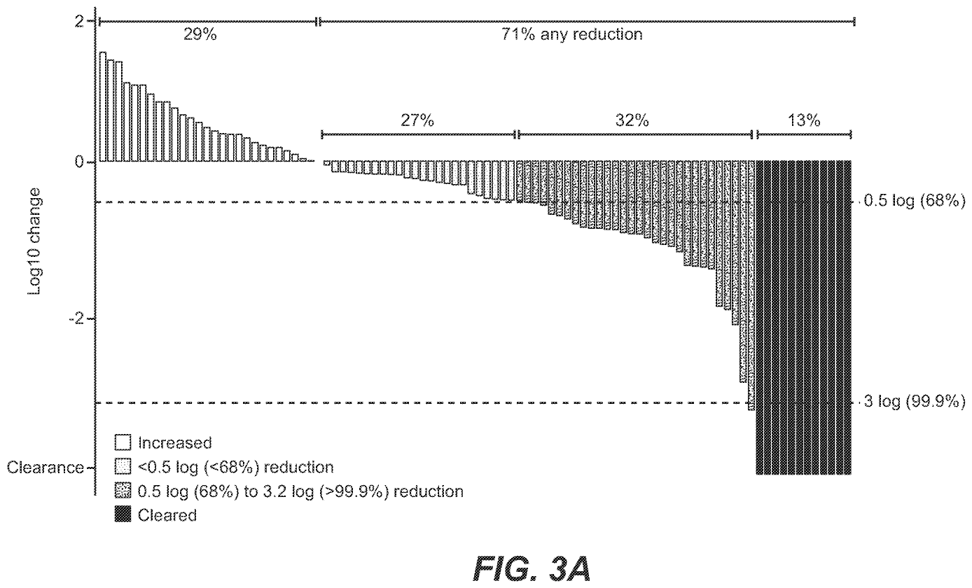

In this patient population with previously treated metastatic uveal melanoma, the median OS was 16.8 months (95% CI: 12.9-21.3) and the median follow-up was 19.5 months. The estimated 1-year and 2-year OS rates were 62% (95% CI: 53-70%) (FIG. 2) and 37% (95% CI: 27-48%), respectively. Longer survival was associated with development of any tumor shrinkage, including PR by RECIST criteria, with most patients (86%) with tebentafusp-induced tumor shrinkage alive at 12 months. Even among patients with tumor growth (≥20% increase from baseline) as best change on treatment, 43% (12/28) were alive at 12 months. In a predefined subgroup analysis, the OS at 1-year was 76% in patients who had previously relapsed (BOR of CR/PR or SD on prior therapy) following immunological checkpoint inhibition compared to 60% in patients refractory (BOR of PD on prior therapy) to prior checkpoint inhibition.

### 7.2.5.4 Early Reduction in ctDNA as a Surrogate Marker for Tebentafusp Activity

ctDNA levels were assessed at baseline and at weeks 5, 9, and 25, after completion of 1, 2, and 6 cycles of treatment, respectively. Of the 127 patients in the trial, 118 (93%) had evaluable serum samples with most (109/118; 92%) determined to have detectable ctDNA at any time point up to and including week 9 (baseline, week 5, week 9). 99 of these 118 patients (84%) had detectable ctDNA at baseline and on-treatment, of whom 94 had variants with a variant allelic frequency (VAF)>0.3 at baseline and were included in the analyses (Table 4). The week 25 timepoint was excluded from the analysis due to limited patient data.

TABLE 4
Variant allele frequency detected at baseline for a targeted panel
of uveal specific mutations for ctDNA mutation profiling (n = 96)

| | | | | Total | | | | |
|---|---|---|---|---|---|---|---|---|
| | | Frequency | | Frequency | Median VAF (IQR | | VAF | VAF |
| Gene | HGVSp | No. (%) | | (%) | range) | | min | max |
| GNAQ | p.Q209L | 9 | (9.38) | 40 | 3.24 | (0.57-12.39) | 0.1 | 29.6 |
| | p.Q209P | 29 | (30.21) | | 7.87 | (1.56-22.07) | 0.1 | 31.4 |
| GNA11 | p.Q209L | 46 | (47.92) | 48 | 3.86 | (0.91-11.47) | 0.0 | 37.0 |
| SF3B1 | p.K700E | 3 | (3.12) | 32 | 0.94 | (0.5-2.88) | 0.1 | 4.8 |
| | p.R625L | 5 | (5.21) | | 0.06 | (0.02-0.11) | 0.0 | 0.2 |
| | p.R625H | 13 | (13.54) | | 8.43 | (2.31-11.13) | 0.1 | 30.4 |
| | p.R625C | 10 | (10.42) | | 0.63 | (0.25-2.19) | 0.1 | 16.2 |
| PLCB4 | p.D630Y | 3 | (3.12) | 4 | 0.07 | (0.06-0.07) | 0.1 | 0.1 |
| | p.D630V | 1 | (1.04) | | 18.25 | (18.25-18.25) | 18.2 | 18.2 |
| CYSLTR2 | p.L129Q | 3 | (3.12) | 3 | 0.91 | (0.62-4.74) | 0.3 | 8.6 |

VAF, variant allele frequency; IQR, interquartile range

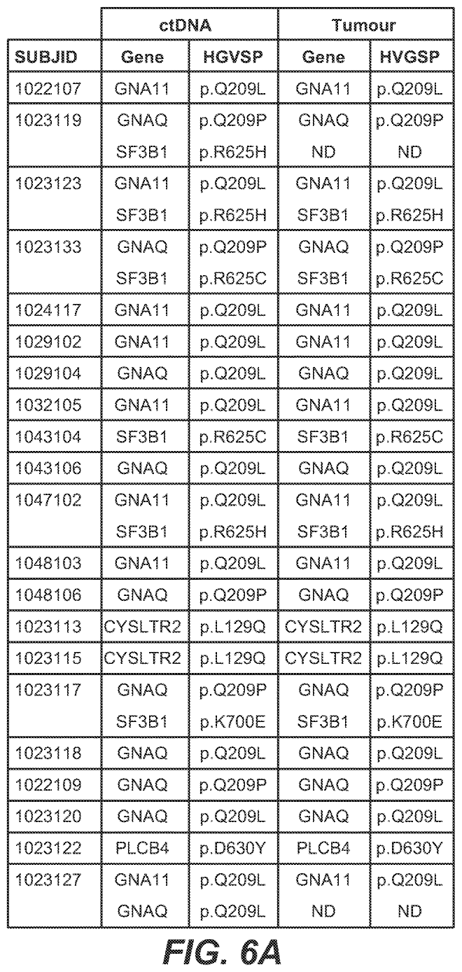

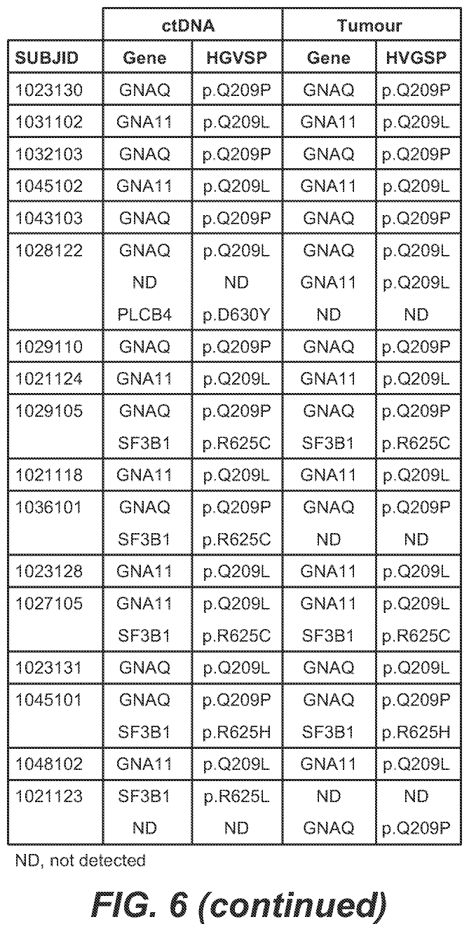

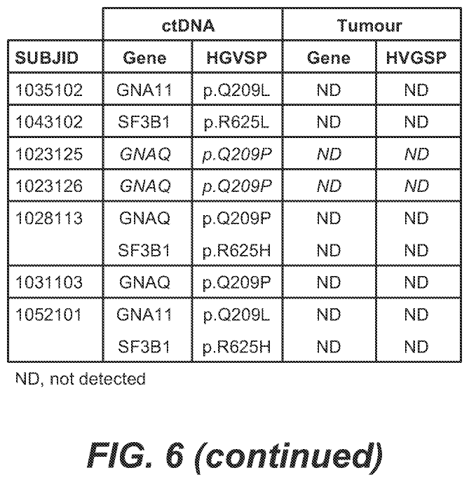

In the subset of patients (n=45) with both baseline ctDNA data and mutational analysis from tumor biopsies, there was excellent concordance: 82% of the known uveal melanoma specific mutations in ctDNA were also found in tumor biopsies; for mutations in GNAQ/GNA11 concordance was 85% (FIG. 6). However, the sensitivity of base calling in the sequencing of tumor biopsies was lower due to a lack of matched normal tissue. For patients with known uveal mutations detected in both tumor biopsies and ctDNA (n=38), concordance was 97%.

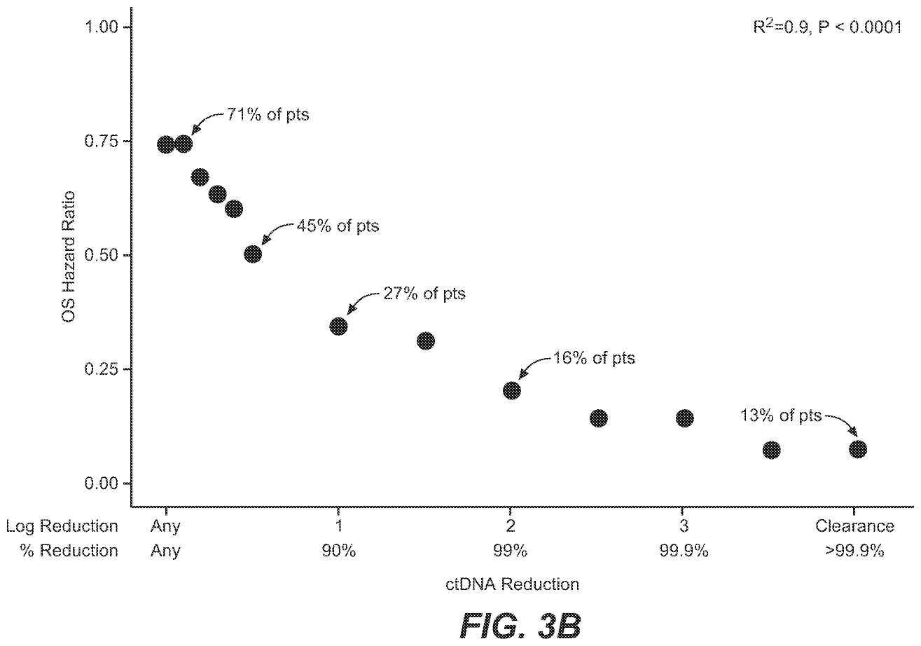

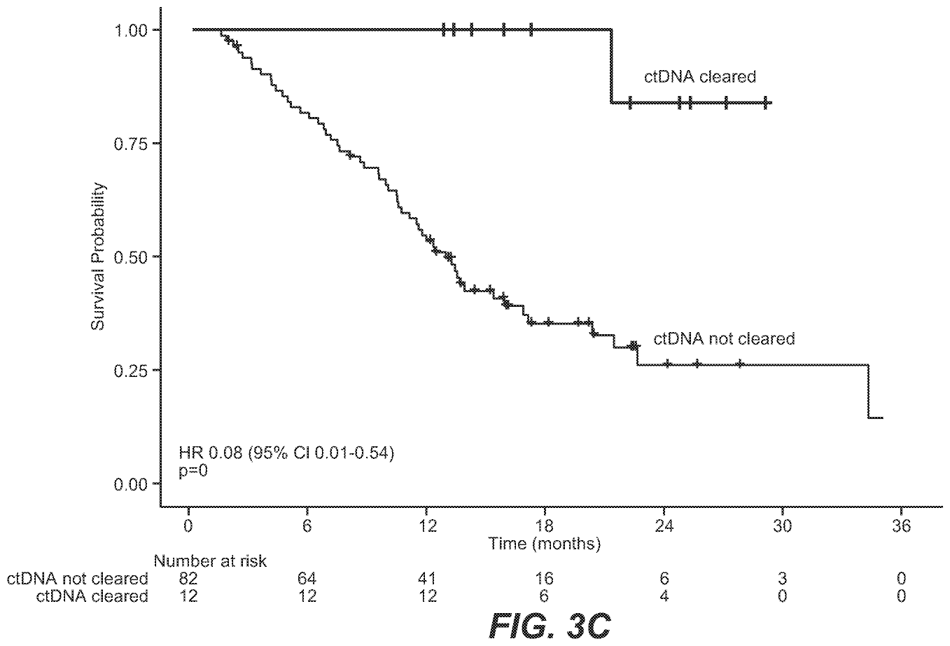

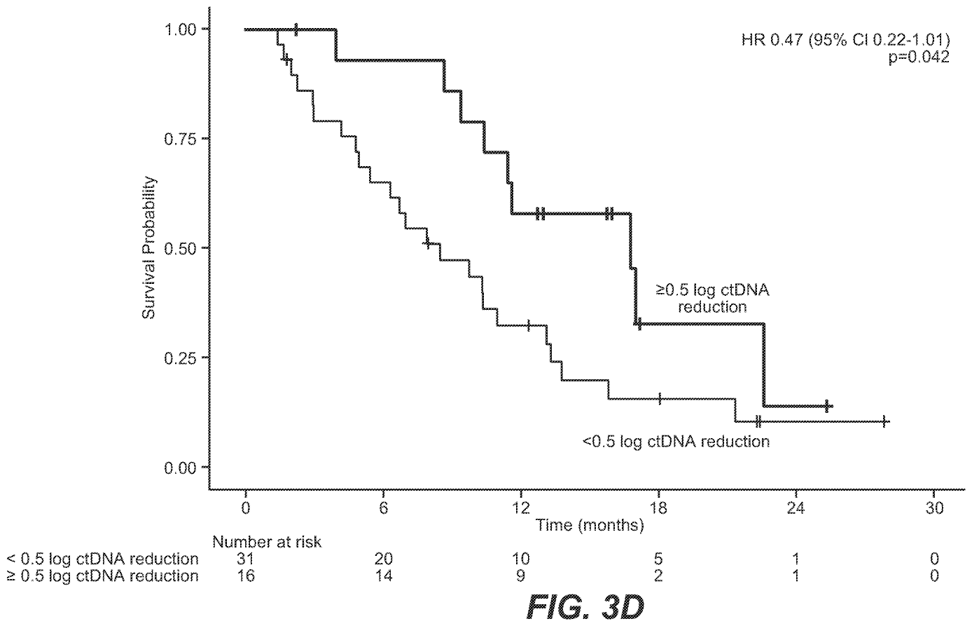

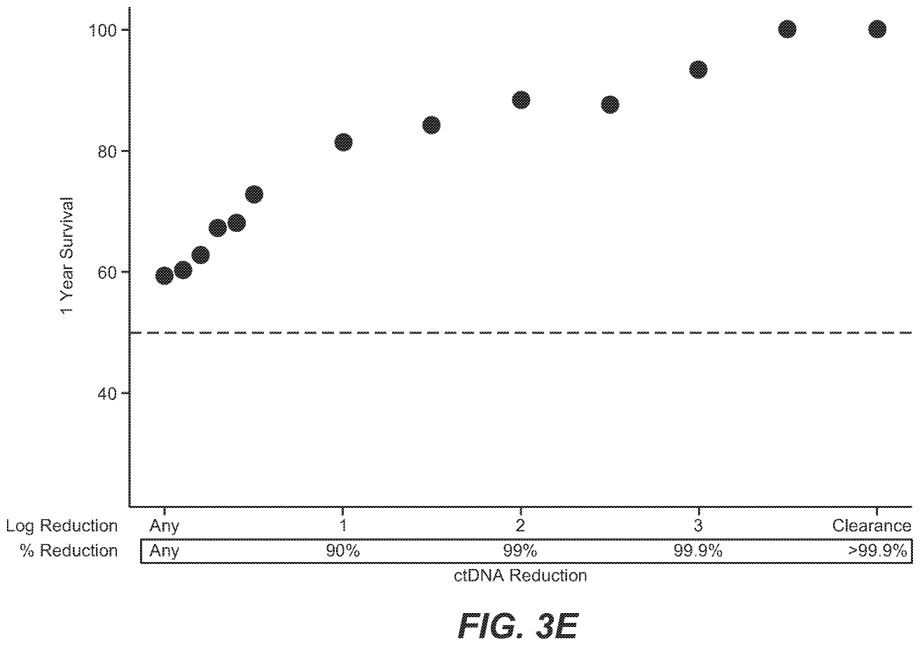

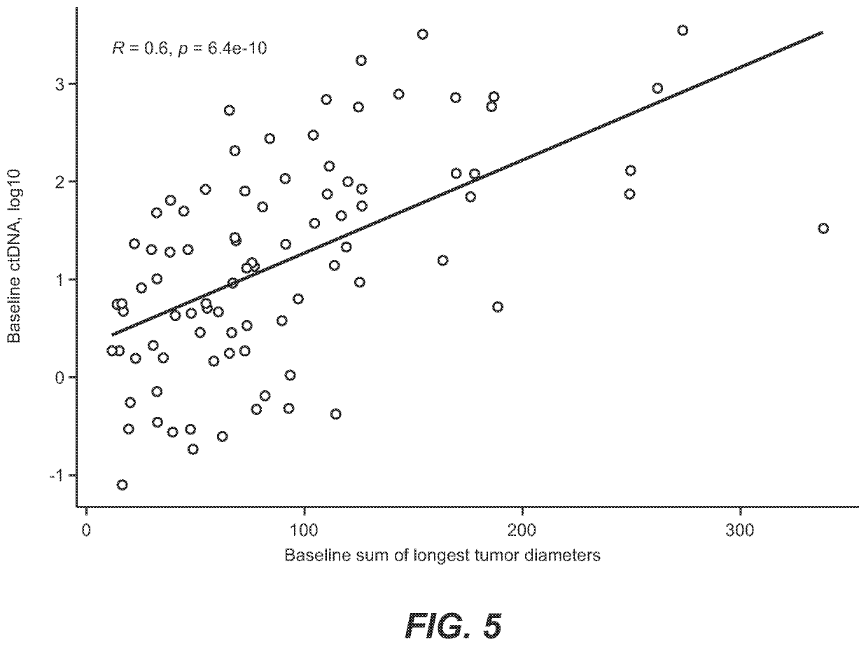

Mean tumor molecules per mL plasma (MTM) detected at baseline was strongly correlated with tumor burden as defined as RECIST sum of longest diameters of target lesions (Spearman's r=0.6, P=6×10⁻¹⁰; FIG. 5). By week 9, 71% (67/94) of patients with baseline and on-treatment MTM measurements had any (>0) ctDNA reduction and 12 patients had complete ctDNA clearance (undetectable) (FIG. 3A). Of those patients with ctDNA clearance, one had a PR, seven had SD, three had PD and one was non-evaluable by RECIST.

The magnitude of ctDNA reduction by week 9 was strongly associated with improvement in overall survival (OS) (R²=0.9, P<0.0001): a 0.1 log reduction was associated with a hazard ratio (HR) of 0.8, while a 1 log reduction was associated with a HR of 0.4, a 2 log reduction was associated with a HR of 0.2, a 3 log reduction was associated with a HR of 0.2, and ctDNA clearance was associated with a HR of 0.1 (FIG. 3B). The 1-year OS rate in patients with ctDNA clearance (n=12) was 100% versus 52% in those with increased ctDNA (n=27) (FIG. 3C). Notably, of the 47 patients with a best radiographic response of PD who were also evaluable for ctDNA, one third (n=16) had a ≥0.5 log reduction in ctDNA by week 9, which was associated with improved OS, in comparison to PD patients with a <0.5 log ctDNA reduction (FIG. 3D).

Evaluation by RECIST v1.1 criteria underestimated the degree of clinical benefit from tebentafusp. While the primary endpoint of ORR was low at 5%, 44% of patients achieved some degree of tumor shrinkage. Over 70% of all patients were treated beyond initial radiographic progression, and nearly half of patients with tumor growth (≥20%) as best response were alive 12 months after commencing treatment with tebentafusp.

Results of this study demonstrate a significant linear relationship between the level of ctDNA reduction and OS. Baseline ctDNA levels correlated with tumor burden and, by week 9 on tebentafusp, more than two-thirds of patients showed some degree of ctDNA reduction, with greater reduction being associated with longer survival (FIG. 3D and FIG. 3E). This association was true even for patients with a best radiographic response of PD, four of whom had complete ctDNA clearance.

These findings show that early reductions in ctDNA, including reductions in ctDNA ascertained at week 5, and at week 9, reflect tebentafusp-related activity in the tumor and correspond to a more precise molecular predictor of clinical response to tebentafusp than traditional radiographic criteria.

### 7.3. Example 2: Phase 1/2 Dose-Escalation Study of IMC-F106C in Subjects with Advanced PRAME-Positive Cancers

### 7.3.1. Study Description and Design

This open-label, phase 1/2, first-in-human study was designed to assess the safety, tolerability, and efficacy of IMC-F106C in HLA-A*02:01-positive subjects with advanced PRAME-positive cancers as a monotherapy and in combination with checkpoint inhibitors (e.g., atezolizumab, pembrolizumab) or in combination with a chemotherapy (e.g., gemcitabine, nab-paclitaxel).

IMC-F106C was administered via weekly (Q1W) IV infusion in 21-day cycles. Each cycle contained a first dose at day 1, a second dose on day 8, and a third dose on day 15. Th duration of IV infusion was typically about 1 hour in Cycles 1 and 2 and about 30 minutes starting at Cycle 3 Day 1.

Safety assessments included physical examination, vital signs, weight, Eastern Cooperative Oncology Group (ECOG) performance status, hematology, chemistry, coagulation, urinalysis, thyroid function, cytokine testing, pregnancy testing, cardiac testing, as well as Adverse Events (AE) collection. AEs were graded according to the National Cancer Institute Common Terminology Criteria for Adverse Events (NCI CTCAE) v5.0 unless otherwise specified.

Tumor response was determined locally according to Response Evaluation Criteria in Solid Tumors (RECIST) v1.1. ctDNA was assessed using one of two defined panels depending on tumor type: a custom panel comprising GNAQ, GNA11, SF3B1, PLCB4, CYSLTR2, and EIF1AX, as described above, or the G360™ panel comprising 73 genes frequently mutated in diverse cancers (Guardant Health, Inc.).

### 7.3.2. Summary of Results

Results of the study demonstrated that IMC-F106C was well-tolerated in human subjects. In particular, a first dose of 6 micrograms on day 1, a second dose of 15 micrograms on day 8, and a third dose of 80 micrograms on day 15 (6/15/80 micrograms) was well tolerated, supporting escalation to higher dosages of, for example, 15/40/160 micrograms and 20/60/240 micrograms or higher.

Administration of IMC-F106C activated T cells. IMC-F106C was well-tolerated with CRS mostly Grade 1, and no Grade ≥3. CRS occurred predominantly during the initial three doses.

IMC-F106C-associated treatment-related adverse events were manageable; no adverse events led to discontinuation of treatment or death. There was a consistent and strong pharmacodynamic biomarker activity observed at a third dose or cohort designated dose (CDD) of 20 micrograms and higher of IMC-F106C.

Treatment with IMC-F106C resulted in durable (9+ months) partial response (PR) according to RECIST criteria across multiple tumor types. In uveal melanoma, PRs were observed in 50% of evaluable patients (3 of 6). In subjects with cutaneous melanoma, all of whom had prior anti-PD1 and ipilumumab treatment, PRs were observed for 33% of evaluable patients (2 of 6). In subjects with platinum-resistant serous ovarian cancer PRs were observed in 50% of evaluable patients (2 of 4). A number of subjects without PR were observed to have long term disease stabilization. Treatment with IMC-F106C also resulted in an observed benefit in disease control, including conversion of stable disease (SD) to PR.

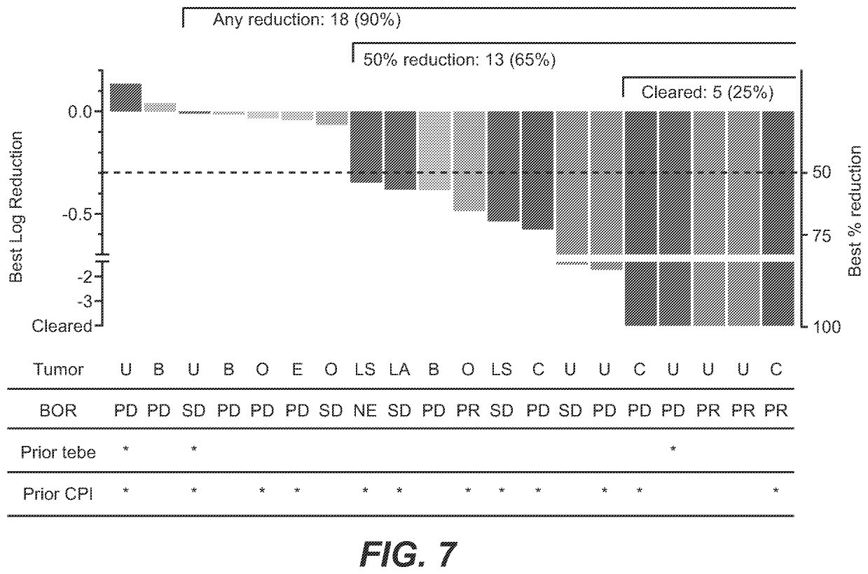

Almost all of the evaluable subjects (18 of 20; 90%), across multiple tumor types, presented with a reduction in ctDNA. Approximately two-thirds of evaluable subjects had at least a 50% reduction in ctDNA levels following treatment with IMC-F106C. 25% of evaluable subjects had ctDNA clearance following treatment with IMC-F106C (FIG. 7).

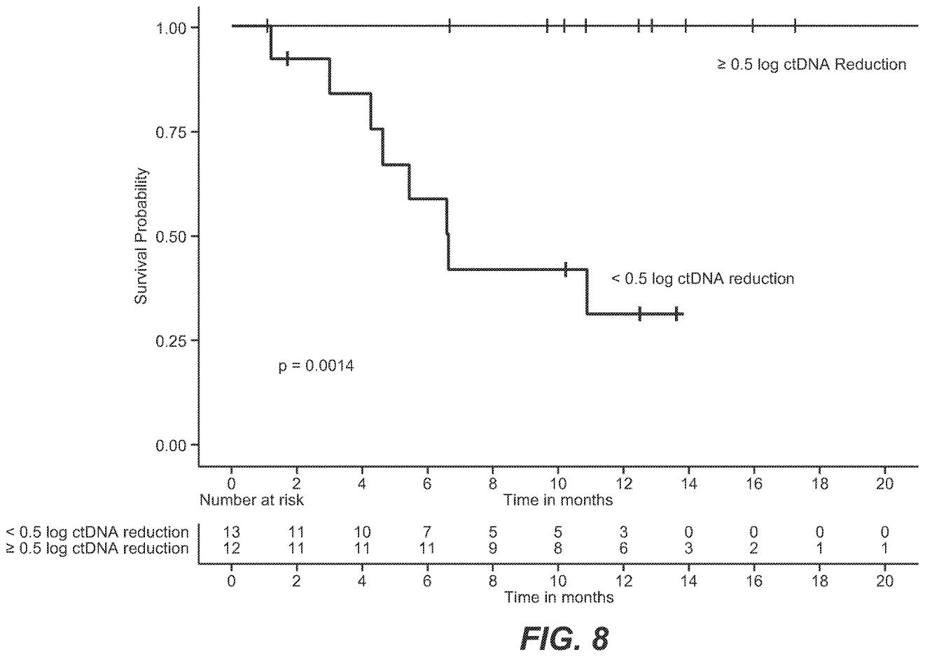

Reduction and clearance of ctDNA were generally observed after one to two months of treatment with IMC-F106C (administered once per week). Four patients with PRs who were evaluated for ctDNA had ≥50% reduction in ctDNA, including three patients that demonstrated ctDNA clearance. One patient with cutaneous melanoma and one patient with uveal melanoma each had ctDNA clearance following IMC-F106C despite having progressive disease (PD) according to RECIST criteria. Correlation between ctDNA reduction and overall survival in subjects treated with IMC-F106C was observed (FIG. 8).

### 8. EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

### 9. SEQUENCES

| SEQ ID NO | DESCRIPTION | SEQUENCE |
|---|---|---|
| SEQ ID NO: | Tebentafusp alpha | AQQGEEDPQALSIQEGENATMNCSYKTSINNLQW |
| 1 | chain | YRQNSGRGLVHLILIRSNEREKHSGRLRVTLDTS |
| | | KKSSSLLITASRAADTASYFCATDGSTPMQFGKG |
| | | TRLSVIANIQKPDPAVYQLRDSKSSDKSVCLFTD |
| | | FDSQTNVSQSKDSDVYITDKCVLDMRSMDFKSNS |
| | | AVAWSNKSDFACANAFNNSIIPEDT |
| SEQ ID NO: | Tebentafusp beta | AIQMTQSPSSLSASVGDRVTITCRASQDIRNYLN |
| 2 | chain | WYQQKPGKAPKLLIYYTSRLESGVPSRFSGSGSG |
| | | TDYTLTISSLQPEDFATYYCQQGNTLPWTFGQGT |
| | | KVEIKGGGGSGGGGSGGGGSGGGGSGGGSEVQLV |
| | | ESGGGLVQPGGSLRLSCAASGYSFTGYTMNWVRQ |
| | | APGKGLEWVALINPYKGVSTYNQKFKDRFTISVD |
| | | KSKNTAYLQMNSLRAEDTAVYYCARSGYYGDSDW |
| | | YFDVWGQGTLVTVSSGGGGSDGGITQSPKYLFRK |
| | | EGQNVTLSCEQNLNHDAMYWYRQDPGQGLRLIYY |
| | | SWAQGDFQKGDIAEGYSVSREKKESFPLTVTSAQ |
| | | KNPTAFYLCASSWGAPYEQYFGPGTRLTVTEDLK |
| | | NVFPPEVAVFEPSEAEISHTQKATLVCLATGFYP |
| | | DHVELSWWVNGKEVHSGVCTDPQPLKEQPALNDS |
| | | RYALSSRLRVSATFWQDPRNHFRCQVQFYGLSEN |
| | | DEWTQDRAKPVTQIVSAEAWGRAD |
| SEQ ID NO: | gp100 peptide | YLEPGPVTA |
| 3 | | |
| SEQ ID NO: | IMC-F106C alpha | GDAKTTQPNSMESNEEEPVHLPCNHSTISGTDYI |
| 4 | chain | HWYRQLPSQGPEYVIHGLTSNVNNRMASLAIAED |
| | | RKSSTLILHRATLRDAAVYYCILILGHSRLGNYI |
| | | ATFGKGTKLSVIPNIQNPDPAVYQLRDSKSSDKS |
| | | VCLFTDFDSQTNVSQSKDSDVYITDKCVLDMRSM |
| | | DFKSNSAVAWSNKSDFACANAENNSIIPEDT |
| SEQ ID NO: | IMC-F106C beta | AIQMTQSPSSLSASVGDRVTITCRASQDIRNYLN |
| 5 | chain | WYQQKPGKAPKLLIYYTSRLESGVPSRFSGSGSG |
| | | TDYTLTISSLQPEDFATYYCQQGNTLPWTFGQGT |
| | | KVEIKGGGGSGGGGSGGGGSGGGGSGGGSEVQLV |
| | | ESGGGLVQPGGSLRLSCAASGYSFTGYTMNWVRQ |
| | | APGKGLEWVALINPYKGVSTYNQKFKDRFTISVD |
| | | KSKNTAYLQMNSLRAEDTAVYYCARSGYYGDSDW |
| | | YFDVWGQGTLVTVSSGGGGSDGGITQSPKYLFRK |
| | | EGQNVTLSCEQNLNHDAMYWYRQDPGQGLRLIYY |
| | | SQIMGDEQKGDIAEGYSVSREKKESFPLTVTSAQ |
| | | KNPTAFYLCASSWWTGGASPIRFGPGTRLTVTED |
| | | LKNVFPPEVAVFEPSEAEISHTQKATLVCLATGF |
| | | YPDHVELSWWVNGKEVHSGVCTDPQPLKEQPALN |
| | | DSRYALSSRLRVSATFWQDPRNHFRCQVQFYGLS |
| | | ENDEWTQDRAKPVTQIVSAEAWGRAD |
| SEQ ID NO: | Peptide target of | SLLQHLIGL |
| 6 | IMC-F106C | |
| | (residues 425-433 | |
| | of full length | |
| | PRAME) | |

What is claimed is:

1. A method of treating a cancer in a subject, comprising:
(a) measuring the circulating tumor DNA (ctDNA) level in the subject prior to administering a T cell therapy to obtain a baseline ctDNA level;
(b) administering to the subject a first plurality of doses of the T cell therapy;
(c) measuring the ctDNA level in the subject following administration of the first plurality of doses of the T cell therapy to obtain a post-treatment ctDNA level; and
(d) administering at least a second plurality of doses of a T cell therapy when the post-treatment ctDNA level is less than or equal to the baseline ctDNA level.

2. The method of claim 1, wherein the subject has a tumor that is larger in size following administration of the first plurality of doses of the T cell therapy compared to the size of the tumor prior to administration of the T cell therapy, as measured using radiographic analysis.

3. The method of claim 1 or claim 2, wherein the cancer is positive for an antigen selected from the group consisting of gp100 and PRAME.

4. The method of any one of the preceding claims, wherein the cancer is selected from the group consisting of: melanoma, ovarian cancer, non-small cell lung adenocarcinoma, non-small cell lung squamous cell carcinoma, and breast cancer.

5. The method of claim 4, wherein the cancer is uveal melanoma or cutaneous melanoma.

6. The method of claim 4, wherein the cancer is triple-negative breast cancer.

7. The method of any one of the preceding claims, wherein the cancer is metastatic.

8. The method of any one of claims 1-7, wherein the subject is determined to have progressive disease (PD) according to RECIST criteria following administration of at least the first plurality of doses of the T cell therapy.

9. The method of any one of claims 1-7, wherein the subject is determined to have stable disease (SD) according to RECIST criteria following administration of at least the first plurality of doses of the T cell therapy.

10. The method of any one of claims 1-7, wherein the subject is determined to have had a partial response (PR) according to RECIST criteria following administration of at least the first plurality of doses of the T cell therapy.

11. The method of any one of the preceding claims, wherein the T cell therapy is tebentafusp.

12. The method of claim 11, wherein the first plurality of doses comprises at least one starting dose of tebentafusp in the range of 10 to 30 micrograms.

13. The method of claim 12, wherein the first plurality of doses further comprises at least one escalated dose of tebentafusp in the range of 20 to 40 micrograms, wherein the escalated dose is higher than the starting dose and is administered after the at least one starting dose.

14. The method of claim 13, wherein the first plurality of doses further comprises at least one continuing dose of tebentafusp of at least 50 micrograms administered after the at least one escalated dose.

15. The method of claim 14, wherein the continuing dose is 68 micrograms.

16. The method of any one of claims 1-10, wherein the T cell therapy is IMC-F106C.

17. The method of claim 16, wherein the first plurality of doses comprises at least one starting dose of IMC-F106C in the range of 5 to 40 micrograms.

18. The method of claim 17, wherein the first plurality of doses further comprises at least one escalated dose of IMC-F106C in the range of 15 to 80 micrograms, wherein the escalated dose is higher than the starting dose and is administered after the at least one starting dose.

19. The method of claim 18, wherein the first plurality of doses further comprises at least one continuing dose of IMC-F106C in the range of 60 to 400 micrograms, wherein the continuing dose is higher than the escalated dose and is administered after the at least one escalated dose.

20. The method of any one of the preceding claims, wherein the first plurality of doses comprises at least one treatment cycle, wherein a treatment cycle consists of once-weekly doses of the T cell therapy for a period of three weeks (21 days) or four weeks (28 days).

21. The method of any one of claims 1-20, wherein the first plurality of doses consists of four doses administered once weekly and wherein the post-treatment ctDNA level is measured at week 5 following administration of the first dose of T cell therapy.

22. The method of any one of claims 1-20, wherein the first plurality of doses consists of eight doses administered once weekly, and wherein the post-treatment ctDNA level is measured at week 9 following administration of the first dose of T cell therapy.

23. The method of any one of claims 1-22, wherein ctDNA is measured by amplifying a panel of genes and then sequencing the amplicons to identify mutations.

24. The method of any one of claims 1-22, wherein ctDNA is measured by amplification of a panel of tumor-specific gene mutations from samples of serum from the subject.

25. The method of claim 24, wherein the amplified ctDNA is assessed to confirm the presence of mutations in a specific group of genes.

26. The method of claim 23 or claim 25, wherein the amplified ctDNA is assessed for mutations in a group of genes consisting of GNAQ, GNA11, SF3B1, PLCB4, CYSLTR2 and EIF1AX.

27. The method of any one of the preceding claims, wherein the post-treatment ctDNA level is reduced by at least 50% from baseline.

28. The method of any one of the preceding claims, wherein the post-treatment ctDNA level is reduced by at least 60% from baseline.

29. The method of any one of the preceding claims, wherein the post-treatment ctDNA level is reduced by at least 65% from baseline.

30. The method of any one of the preceding claims, wherein the post-treatment ctDNA level is reduced by about 68% from baseline.

31. A method of treating melanoma in a subject, comprising:
administering to the subject a first dose of 20 micrograms tebentafusp, a second dose of 30 micrograms tebentafusp, a third dose of at least 50 micrograms of tebentafusp, and a fourth dose of at least 50 micrograms of tebentafusp, and as long as ctDNA levels remain stable or are reduced as compared to a baseline ctDNA level, subsequent weekly doses of at least 50 micrograms tebentafusp, wherein doses are administered every 6 to 8 days.

32. The method of claim 31, wherein the melanoma is cutaneous melanoma or uveal melanoma.

33. A method of treating a PRAME positive cancer in a subject, comprising:
administering to the subject a first dose of IMC-F106C in the range of 5 to 40 micrograms, a second dose of IMC-F106C in the range of 15 to 80 micrograms, and a third dose of IMC-F106C in the range of 60 to 400 micrograms, and as long as ctDNA levels remain stable or are reduced as compared to a baseline ctDNA level, subsequent weekly doses of IMC-F106C in the range of 60 to 400 micrograms, wherein the second dose is higher than the first dose and the third dose is higher than the second dose and wherein the doses are administered every 6 to 8 days.

34. The method of claim 33, wherein the PRAME positive cancer is selected from the group consisting of: melanoma, ovarian cancer, non-small cell lung adenocarcinoma, non-small cell lung squamous cell carcinoma, and breast cancer.

35. The method of claim 34, wherein the PRAME positive cancer is uveal melanoma or cutaneous melanoma.

36. The method of claim 34, wherein the PRAME positive cancer is triple-negative breast cancer.

37. A method of treating a subject who has or is suspected of having uveal melanoma, comprising:
(a) administering to the subject a first plurality of doses of tebentafusp or IMC-F106C;
(b) determining that the subject has a reduction in ctDNA from baseline following administration of the first plurality of doses of tebentafusp or IMC-F106C, wherein the reduction in ctDNA is determined by evaluating variant allele frequency in a panel of mutations; and
(c) administering to the subject at least a second plurality of doses of tebentafusp or IMC-F106C.

38. The method of claim 37, wherein the panel of mutations comprises GNAQ (Q209L/P); GNA11 (Q209L); SF3B1 (K700E, R625L/H/C); PLCB4 (D630N/Y/V); CYSLTR2 (L129Q); and EIF1AX (G15D).

39. A kit, comprising:
(a) at least one solution for use in processing a sample obtained from a human subject, wherein the sample comprises cell-free DNA; and
(b) nucleotide primers for evaluating variant allele frequency in a panel of mutations comprising GNAQ (Q209L/P); GNA11 (Q209L); SF3B1 (K700E, R625L/H/C); PLCB4 (D630N/Y/V); CYSLTR2 (L129Q); and EIF1AX (G15D).
